SEQUENCE LISTING (48 sequences)

SEQ ID NO: 1 - protein, 241 aa, synthetic construct
  REGION 1..241: note = Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVL

SEQ ID NO: 2 - protein, 241 aa, synthetic construct
  REGION 1..241: note = Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVL

SEQ ID NO: 3 - protein, 460 aa, synthetic construct
  REGION 1..460: note = Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 318 aa, synthetic construct
  REGION 1..318: note = Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVRVKFSRSADAPA

SEQ ID NO: 5 - protein, 459 aa, synthetic construct
  REGION 1..459: note = Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 501 aa, synthetic construct
  REGION 1..501: note = Synthetic polypeptide
MLLLVTSLLLCELPHPAFLLIPGVHSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 456 aa, synthetic construct
  REGION 1..456: note = Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 314 aa, synthetic construct
  REGION 1..314: note = Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVRVKFSRSADAPA

SEQ ID NO: 9 - protein, 455 aa, synthetic construct
  REGION 1..455: note = Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 497 aa, synthetic construct
  REGION 1..497: note = Synthetic polypeptide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1383 bp, synthetic construct
  misc_feature 1..1383: note = Synthetic polynucleotide
  source 1..1383: mol_type = other DNA
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtga

SEQ ID NO: 12 - DNA, 957 bp, synthetic construct
  misc_feature 1..957: note = Synthetic polynucleotide
  source 1..957: mol_type = other DNA
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtccgcgtgaagttttctcgaagtgccgatgctcctgcatga

SEQ ID NO: 13 - DNA, 1380 bp, synthetic construct
  misc_feature 1..1380: note = Synthetic polynucleotide
  source 1..1380: mol_type = other DNA
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattccctgctggtcactgtggccttcatcatcttctgggtgcggagcaagcggagccggctgctgcactctgactacatgaacatgactccacggagacccggccctacccggaaacattatcagccctacgccccacccagagattttgccgcttataggtccagggtgaagttttctcgcagtgcagatgcccctgcttatcagcagggacagaatcagctgtacaacgagctgaatctgggcaggcgcgaggaatacgacgtgctggataagcgacggggcagagaccccgaaatgggagggaagcccagaaggaaaaaccctcaggaggggctgtataatgaactgcagaaggacaaaatggcagaggcctacagtgaaatcgggatgaagggagagcgccgacggggaaaaggccacgatggactgtatcagggcctgtctactgccaccaaggacacctacgatgccctgcacatgcaggctctgcctccacgctga

SEQ ID NO: 14 - DNA, 1506 bp, synthetic construct
  misc_feature 1..1506: note = Synthetic polynucleotide
  source 1..1506: mol_type = other DNA
atgctgctgctggtgacaagcctgctgctgtgcgaactgccccatcccgccttcctgctgattcctggtgtacactccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattccctgctggtcactgtggccttcatcatcttctgggtgcggagcaagcggagccggctgctgcactctgactacatgaacatgactccacggagacccggccctacccggaaacattatcagccctacgccccacccagagattttgccgcttataggtccaagcgcggccgaaagaaactgctgtacatcttcaaacagcccttcatgagacccgtccagacaactcaggaggaagacggctgcagctgtaggttccccgaggaagaggaagggggatgtgagctgagggtgaagttttctcgcagtgcagatgcccctgcttatcagcagggacagaatcagctgtacaacgagctgaatctgggcaggcgcgaggaatacgacgtgctggataagcgacggggcagagaccccgaaatgggagggaagcccagaaggaaaaaccctcaggaggggctgtataatgaactgcagaaggacaaaatggcagaggcctacagtgaaatcgggatgaagggagagcgccgacggggaaaaggccacgatggactgtatcagggcctgtctactgccaccaaggacacctacgatgccctgcacatgcaggctctgcctccacgctga

SEQ ID NO: 15 - DNA, 1371 bp, synthetic construct
  misc_feature 1..1371: note = Synthetic polynucleotide
  source 1..1371: mol_type = other DNA
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtga

SEQ ID NO: 16 - DNA, 945 bp, synthetic construct
  misc_feature 1..945: note = Synthetic polynucleotide
  source 1..945: mol_type = other DNA
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttactccctgctggtcactgtggccttcatcatcttctgggtgcgggtgaagttttctcgcagtgccgacgctcccgcatga

SEQ ID NO: 17 - DNA, 1368 bp, synthetic construct
  misc_feature 1..1368: note = Synthetic polynucleotide
  source 1..1368: mol_type = other DNA
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttattccctgctggtcacagtggccttcatcatcttctgggtgcggagcaagcggagccggctgctgcactctgactacatgaacatgaccccccggagacccggccctacaagaaagcattatcagccttacgccccacccagggacttcgcagcttatcgctcccgagtgaaattttctcgcagtgcagatgcccccgcttatcagcagggccagaatcagctgtacaacgagctgaatctggggaggcgcgaggaatacgacgtgctggataagcgacggggccgggaccccgaaatgggaggaaagcctagaaggaaaaacccacaggagggcctgtataatgaactgcagaaggacaaaatggcagaggcctacagcgaaatcggaatgaagggagagcgccgacggggcaaaggacacgatggcctgtatcaggggctgagcaccgccacaaaggacacctacgatgccctgcacatgcaggctctgcctccacgctga

SEQ ID NO: 18 - DNA, 1494 bp, synthetic construct
  misc_feature 1..1494: note = Synthetic polynucleotide
  source 1..1494: mol_type = other DNA
atgctgctgctggtgacaagcctgctgctgtgcgaactgccccatcccgccttcctgctgattcctgaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctggagagcaaatatggaccaccatgccctccatgtcctttttgggtcctggtggtcgtgggaggcgtgctggcatgttattccctgctggtcactgtggccttcatcatcttctgggtgcggagcaagcggagccggctgctgcactctgactacatgaacatgactccacggagacccggccctacccggaaacattatcagccctacgccccacccagagattttgccgcttataggtccaagcgcggccgaaagaaactgctgtacatcttcaaacagcccttcatgagacccgtccagacaactcaggaggaagacggctgcagctgtaggttccccgaggaagaggaagggggatgtgagctgagggtgaagttttctcgcagtgcagatgcccctgcttatcagcagggacagaatcagctgtacaacgagctgaatctgggcaggcgcgaggaatacgacgtgctggataagcgacggggcagagaccccgaaatgggagggaagcccagaaggaaaaaccctcaggaggggctgtataatgaactgcagaaggacaaaatggcagaggcctacagtgaaatcgggatgaagggagagcgccgacggggaaaaggccacgatggactgtatcagggcctgtctactgccaccaaggacacctacgatgccctgcacatgcaggctctgcctccacgctga

SEQ ID NO: 19 - protein, 456 aa, synthetic construct
  REGION 1..456: note = Synthetic polynucleotide
MLLLVTSLLLCELPHPAFLLIPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSDKTHTCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 497 aa, synthetic construct
  REGION 1..497: note = Synthetic polynucleotide
MLLLVTSLLLCELPHPAFLLIPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSDKTHTCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 457 aa, synthetic construct
  REGION 1..457: note = Synthetic polynucleotide
MLLLVTSLLLCELPHPAFLLIPQVQLVQSGGGVVQPGRSLRLSCAASGFTFSSYGLHWVRQAPGKGLEWVAAISYDGSKKYYADSVKGRLTISRDNSKNTLYLQMNSLRPDDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 1371 bp, synthetic construct
  misc_feature 1..1371: note = Synthetic polynucleotide
  source 1..1371: mol_type = other DNA
atgctgctgctggtgacaagcctgctgctgtgcgaactgccccatcccgccttcctgctgattcctgatgtcgtgatgacgcagagccctctctctcttcccgttacccctggtgaacccgcatcaataagttgccgctccagtcaatcacttgtacattcaaatcgcaatacctacctgcactggtatttgcagaagccgggacaatcccctcaattgttgatatataaggtatccaatcgcttttctggagttcctgatagattcagcggatccgggtctggtactgatttcactctgaaaatatccagggtcgaagctgaggacgtaggcgtatattattgctctcagaacacgcatgtcccgccgactttcggccagggcactaaacttgagatcaagggtggggggggcagcggtggtggaggctctggtggaggagggagccaggtccaactcgttcaaagtggcgcagaggtcaaaaagccaggcgcgagcgttaaagtatcatgtaaggccagcggttatactttcactgattatgaaatgcactgggtgcgacaagcccccgggcaaggtcttgagtggatgggtgcacttgatccaaaaactggggatactgcctatagccagaaattcaaagggcgcgtcacactcactgccgacaaaagtacgagcacagcttatatggaattgagttcactgacgagcgaggatacggcagtttattactgtacgcgcttctactcttacacttattgggggcaaggcactttggttactgtgtcctctgacaagacccatacgtgtccaccgtgtcccttctgggtattggttgtggtcggcggtgtccttgcttgttacagccttctcgtgacagtcgcattcataattttttgggtgaaaagaggtcggaaaaagttgctgtatattttcaaacaaccctttatgagacctgtacaaacgactcaggaagaggatggttgtagttgcaggtttccggaggaggaggaaggtgggtgcgaactgcgggtgaaatttagtagaagcgctgacgcaccagcttaccaacaaggacagaaccaattgtacaacgagcttaacttgggtaggagggaggaatatgatgtactggacaaaaggcgaggtcgcgatccggaaatgggaggcaagccacagcgccggaaaaacccgcaggaaggcttgtacaacgaacttcagaaagataaaatggcagaagcatactccgaaatagggatgaaaggtgaacggcggcgaggcaagggccacgacggtctgtaccaagggttgtcaacggcaactaaagacacgtatgatgcacttcatatgcaagctctgccacccaggtga

SEQ ID NO: 23 - DNA, 1494 bp, synthetic construct
  misc_feature 1..1494: note = Synthetic polynucleotide
  source 1..1494: mol_type = other DNA
atgctgctgctggtgacaagcctgctgctgtgcgaactgccccatcccgccttcctgctgattcctgatgtcgtgatgacgcagagccctctctctcttcccgttacccctggtgaacccgcatcaataagttgccgctccagtcaatcacttgtacattcaaatcgcaatacctacctgcactggtatttgcagaagccgggacaatcccctcaattgttgatatataaggtatccaatcgcttttctggagttcctgatagattcagcggatccgggtctggtactgatttcactctgaaaatatccagggtcgaagctgaggacgtaggcgtatattattgctctcagaacacgcatgtcccgccgactttcggccagggcactaaacttgagatcaagggtggggggggcagcggtggtggaggctctggtggaggagggagccaggtccaactcgttcaaagtggcgcagaggtcaaaaagccaggcgcgagcgttaaagtatcatgtaaggccagcggttatactttcactgattatgaaatgcactgggtgcgacaagcccccgggcaaggtcttgagtggatgggtgcacttgatccaaaaactggggatactgcctatagccagaaattcaaagggcgcgtcacactcactgccgacaaaagtacgagcacagcttatatggaattgagttcactgacgagcgaggatacggcagtttattactgtacgcgcttctactcttacacttattgggggcaaggcactttggttactgtgtcctctgacaagacccatacgtgtccaccgtgtcccttctgggtattggttgtggtcggcggtgtccttgcttgttacagccttctcgtgacagtcgcattcataattttttgggtgcggagcaagcggagccggctgctgcactctgactacatgaacatgactccacggagacccggccctacccggaaacattatcagccctacgccccacccagagattttgccgcttataggtccaaaagaggtcggaaaaagttgctgtatattttcaaacaaccctttatgagacctgtacaaacgactcaggaagaggatggttgtagttgcaggtttccggaggaggaggaaggtgggtgcgaactgcgggtgaaatttagtagaagcgctgacgcaccagcttaccaacaaggacagaaccaattgtacaacgagcttaacttgggtaggagggaggaatatgatgtactggacaaaaggcgaggtcgcgatccggaaatgggaggcaagccacagcgccggaaaaacccgcaggaaggcttgtacaacgaacttcagaaagataaaatggcagaagcatactccgaaatagggatgaaaggtgaacggcggcgaggcaagggccacgacggtctgtaccaagggttgtcaacggcaactaaagacacgtatgatgcacttcatatgcaagctctgccacccaggtga

SEQ ID NO: 24 - DNA, 1374 bp, synthetic construct
  misc_feature 1..1374: note = Synthetic polynucleotide
  source 1..1374: mol_type = other DNA
atgctgctgctggtgacaagcctgctgctgtgcgaactgccccatcccgccttcctgctgattcctcaggtccagcttgtgcaaagcggaggaggagtggtacagcctggccgctctttgagactgtcttgtgcggccagtggatttacattctcttcttatgggttgcattgggtcagacaagcaccgggcaaaggattggaatgggtcgcggccattagctatgatggctcaaagaaatattatgccgattccgtaaaagggaggttgacaataagccgggataacagcaagaacactttgtatcttcagatgaatagcctccgaccggacgacacggcactgtatttttgcgcacgcgggtggtttgtagaacccctgagttggggacaaggtactcttgtcacggtatcttctggcggaggtgggagtggtgggggtggcagtggcgggggtgggtcacaaagcgtgcttacacaacctccttctgcgagcggaactccgggacaacgggttacgatttcatgctccggctcaagtagcaatataggatcaaatacagtgaattggtatcaacaactccctggcacagcgcccaagctgctgatctactctaataaccagaggccgagtggtgtgccagataggttcagtggctctaaatcaggtactagcgcgagcctcgccatttcaggacttcaatcagaggatgaagcggactactactgtgccgcgtgggatgattcacttaatggatatgttttcgggaccggaacaaaattgacggtattggagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggtga

SEQ ID NO: 25 - protein, 456 aa, synthetic construct
  REGION 1..456: note = Synthetic polynucleotide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 1368 bp, synthetic construct
  misc_feature 1..1368: note = Synthetic polynucleotide
  source 1..1368: mol_type = other DNA
atgctgctgctggtgacaagcctgctgctgtgcgaactgccccatcccgccttcctgctgattcctgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccagg

SEQ ID NO: 27 - protein, 116 aa, synthetic construct
  REGION 1..116: note = Synthetic polynucleotide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSS

SEQ ID NO: 28 - protein, 110 aa, synthetic construct
  REGION 1..110: note = Synthetic polynucleotide
SYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVL

SEQ ID NO: 29 - protein, 116 aa, synthetic construct
  REGION 1..116: note = Synthetic polynucleotide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSS

SEQ ID NO: 30 - protein, 110 aa, synthetic construct
  REGION 1..110: note = Synthetic polynucleotide
QSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVL

SEQ ID NO: 31 - protein, 242 aa, synthetic construct
  REGION 1..242: note = Synthetic polynucleotide
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 32 - protein, 242 aa, synthetic construct
  REGION 1..242: note = Synthetic polynucleotide
QVQLVQSGGGVVQPGRSLRLSCAASGFTFSSYGLHWVRQAPGKGLEWVAAISYDGSKKYYADSVKGRLTISRDNSKNTLYLQMNSLRPDDTALYFCARGWFVEPLSWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGTGTKLTVL

SEQ ID NO: 33 - DNA, 723 bp, synthetic construct
  misc_feature 1..723: note = Synthetic polynucleotide
  source 1..723: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtccta

SEQ ID NO: 34 - DNA, 723 bp, synthetic construct
  misc_feature 1..723: note = Synthetic polynucleotide
  source 1..723: mol_type = other DNA
gaggtccagctgctggagagcggaggaggactggtgcagcctggaggaagtctgcgactgtcatgcgccgctagcggcttcaccttcagctcctatgcaatgagctgggtgcgacaggcaccaggcaaggggctggagtgggtctccgctatctccggctctggaggctctacttactatgcagacagtgtgaaggggcggttcacaatctccagagataactctaagaacactctgtacctgcagatgaactctctgagagctgaggacaccgcagtgtactattgcgccaagggcaaaaggtactttgattattggggacagggcactatggtgaccgtctctagtggaggaggaggaagcggaggaggaggatccggcggaggaggcagtcagtcagtgctgacacagccacctagcgcctccggaaccccaggacagcgggtcacaatctcttgtagtgggggatcaagcgacattgggagcaacaccgtgaattggtatcagcagctgcctggaacagctccaaagctgctgatctactataacaatcagaggccctccggcgtccctgatcgcttctcaggcagcaaatccgggacttctgcaagtctggccattagtggcctgcagtcagaggacgaagccgattactattgtgctacctgggacgataggatgtactctcccgtgttcggcgggggaacaaagctgactgtcctg

SEQ ID NO: 35 - DNA, 726 bp, synthetic construct
  misc_feature 1..726: note = Synthetic polynucleotide
  source 1..726: mol_type = other DNA
gatgtcgtgatgacgcagagccctctctctcttcccgttacccctggtgaacccgcatcaataagttgccgctccagtcaatcacttgtacattcaaatcgcaatacctacctgcactggtatttgcagaagccgggacaatcccctcaattgttgatatataaggtatccaatcgcttttctggagttcctgatagattcagcggatccgggtctggtactgatttcactctgaaaatatccagggtcgaagctgaggacgtaggcgtatattattgctctcagaacacgcatgtcccgccgactttcggccagggcactaaacttgagatcaagggtggggggggcagcggtggtggaggctctggtggaggagggagccaggtccaactcgttcaaagtggcgcagaggtcaaaaagccaggcgcgagcgttaaagtatcatgtaaggccagcggttatactttcactgattatgaaatgcactgggtgcgacaagcccccgggcaaggtcttgagtggatgggtgcacttgatccaaaaactggggatactgcctatagccagaaattcaaagggcgcgtcacactcactgccgacaaaagtacgagcacagcttatatggaattgagttcactgacgagcgaggatacggcagtttattactgtacgcgcttctactcttacacttattgggggcaaggcactttggttactgtgtcctct

SEQ ID NO: 36 - DNA, 726 bp, synthetic construct
  misc_feature 1..726: note = Synthetic polynucleotide
  source 1..726: mol_type = other DNA
caggtccagcttgtgcaaagcggaggaggagtggtacagcctggccgctctttgagactgtcttgtgcggccagtggatttacattctcttcttatgggttgcattgggtcagacaagcaccgggcaaaggattggaatgggtcgcggccattagctatgatggctcaaagaaatattatgccgattccgtaaaagggaggttgacaataagccgggataacagcaagaacactttgtatcttcagatgaatagcctccgaccggacgacacggcactgtatttttgcgcacgcgggtggtttgtagaacccctgagttggggacaaggtactcttgtcacggtatcttctggcggaggtgggagtggtgggggtggcagtggcgggggtgggtcacaaagcgtgcttacacaacctccttctgcgagcggaactccgggacaacgggttacgatttcatgctccggctcaagtagcaatataggatcaaatacagtgaattggtatcaacaactccctggcacagcgcccaagctgctgatctactctaataaccagaggccgagtggtgtgccagataggttcagtggctctaaatcaggtactagcgcgagcctcgccatttcaggacttcaatcagaggatgaagcggactactactgtgccgcgtgggatgattcacttaatggatatgttttcgggaccggaacaaaattgacggtattg

SEQ ID NO: 37 - protein, 10 aa, synthetic construct
  REGION 1..10: note = Synthetic polynucleotide
GFTFSSYAMS

SEQ ID NO: 38 - protein, 17 aa, synthetic construct
  REGION 1..17: note = Synthetic polynucleotide
AISGSGGSTYYADSVKG

SEQ ID NO: 39 - protein, 7 aa, synthetic construct
  REGION 1..7: note = Synthetic polynucleotide
GKRYFDY

SEQ ID NO: 40 - protein, 13 aa, synthetic construct
  REGION 1..13: note = Synthetic polynucleotide
SGGSSNIGSNTVN

SEQ ID NO: 41 - protein, 7 aa, synthetic construct
  REGION 1..7: note = Synthetic polynucleotide
FNNQRPS

SEQ ID NO: 42 - protein, 11 aa, synthetic construct
  REGION 1..11: note = Synthetic polynucleotide
VAWDDSLNAPV

SEQ ID NO: 43 - protein, 13 aa, synthetic construct
  REGION 1..13: note = Synthetic polynucleotide
SGGSSDIGSNTVN

SEQ ID NO: 44 - protein, 7 aa, synthetic construct
  REGION 1..7: note = Synthetic polynucleotide
YNNQRPS

SEQ ID NO: 45 - protein, 11 aa, synthetic construct
  REGION 1..11: note = Synthetic polynucleotide
ATWDDRMYSPV

SEQ ID NO: 46 - DNA, 2013 bp, synthetic construct
  misc_feature 1..2013: note = Synthetic polynucleotide
  source 1..2013: mol_type = other DNA
atgctgctgctggtgacaagcctgctgctgtgcgaactgccccatcccgccttcctgctgattcctgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggaaagcgatactttgactactggggccaggggacaatggtcaccgtctcgagtggtggggggggcagcggtggtggaggctctggtggaggagggagctcctatgagctgactcagccaccctcagcgtctgggacccccgggcagagggtcaccatctcttgttctggaggcagctccaacatcggaagtaatactgtaaactggttccggcagctcccaggaacggcccccaaactcctcgtttattttaataatcagcgaccctcaggggtccctgaccgattctctggctccaagtctggcacctcggcctccctggccatcggtgggctccagtctgacgatgaggctgactattactgtgtagcatgggatgactctctgaatgctccggtgttcggcggagggaccaaggtcaccgtcctagagagcaaatatggaccaccatgccctccatgtcctttttgggtcctggtggtcgtgggaggcgtgctggcatgttattctctgctggtcacagtggctttcatcatcttctgggtcaagcgaggccggaagaaactgctgtacatcttcaaacagccttttatgcgcccagtgcagacaactcaggaggaagacggctgctcttgtcggttccccgaggaagaggaagggggatgtgagctgcgcgtgaagttttctcgaagtgccgatgctcctgcatatcagcagggacagaaccagctgtacaacgagctgaatctgggccggagagaggaatacgacgtgctggataagaggcgcggcagagacccagaaatgggcgggaagccacgacggaaaaacccccaggaggggctgtataatgaactgcagaaggacaaaatggccgaggcttacagcgaaatcgggatgaagggagagagaaggcgcggaaaaggccacgatggactgtatcagggcctgagcactgccaccaaggacacctacgatgctctgcacatgcaggcactgccacccaggggtagcggcgagggcagaggaagtcttctaacatgcggtgacgtggaggagaatcccggccctatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcag

SEQ ID NO: 47 - protein, 671 aa, synthetic construct
  REGION 1..671: note = Synthetic polynucleotide
MLLLVTSLLLCELPHPAFLLIPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSSGGGGSGGGGSGGGGSSYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVLESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 48 - protein, 15 aa, synthetic construct
  REGION 1..15: note = Synthetic polynucleotide
  SITE 1..15: note = This sequence may encompass 1-3 "Gly Gly Gly Gly Ser" repeating units
  REGION 1..15: note = See specification as filed for detailed description of substitutions and preferred embodiments
GGGGSGGGGSGGGGS

(19) United States
(12) Patent Application Publication
(10) Pub. No.: US 2024/0158469 A1
(43) Pub. Date: May 16, 2024

(54) COMPOSITIONS AND METHODS OF TREATING CANCER WITH CHIMERIC ANTIGEN RECEPTORS

(71) Applicant: ASTRAZENECA AB, SODERTALJE (SE)

(72) Inventors: GORDON MOODY, GAITHERSBURG, MD (US); MARIA LETIZIA GIARDINO TORCHIA, GAITHERSBURG, MD (US); MICHAEL GLEN OVERSTREET, GAITHERSBURG, MD (US); RYAN GILBRETH, GAITHERSBURG, MD (US)

(21) Appl. No.: 18/421,142

(22) Filed: Jan. 24, 2024

Related U.S. Application Data
(62) Division of application No. 17/237,498, filed on Apr. 22, 2021.
(60) Provisional application No. 63/014,831, filed on Apr. 24, 2020.

(51) Int. Cl. C07K 14/71; A61K 35/17; A61K 38/17; A61K 39/00; A61K 39/395; A61K 45/06; A61P 35/00; C07K 14/705; C07K 14/715; C07K 14/725; C07K 16/28; C07K 16/30; C07K 16/32

(52) U.S. Cl. CPC C07K 14/71; A61K 35/17; A61K 38/177; A61K 38/1774; A61K 38/179; A61K 39/3955; A61K 39/4611; A61K 39/4613; A61K 39/4631; A61K 39/464474; A61K 45/06; A61P 35/00; C07K 14/7051; C07K 14/70521; C07K 14/70567; C07K 14/70578; C07K 14/7151; C07K 14/7153; C07K 16/2803; C07K 16/2827; C07K 16/283; C07K 16/2836; C07K 16/2866; C07K 16/2896; C07K 16/30; C07K 16/3007; C07K 16/303; C07K 16/3092; C07K 16/32; A61K 2239/21; C07K 2317/53; C07K 2317/55; C07K 2317/622; C07K 2319/02; C07K 2319/03; C07K 2319/30; C07K 2319/32

(57) ABSTRACT

This disclosure relates to compositions and methods for treating cancer using armored chimeric antigen receptor cells.

### SEQUENCE LISTING

The instant application contains a Sequence Listing which has been submitted electronically in ASCII format and is hereby incorporated by reference in its entirety. Said ASCII copy, created on Jan. 23, 2024, is named CARTGPC(TGF)-WO-PCT_SL.xml and is 89,931 bytes in size.

### CROSS-REFERENCE TO RELATED APPLICATIONS

This application is a divisional of U.S. patent application Ser. No. 17/237,498, which has a filing date of Apr. 22, 2021, which claims priority to U.S. Provisional Application No. 63/014,831, filed Apr. 24, 2020, all of which are incorporated herein by reference in their entirety.

### BACKGROUND OF DISCLOSURE

### Field of Invention

This disclosure relates to treatment of cancer using chimeric antigen receptor T cells.

### Technical Background

### 1. Chimeric Antigen Receptor T Cell Therapy

Chimeric antigen receptor (CAR) T cell therapy is a specific form of cell-based immunotherapy that uses engineered T cells to fight cancer. In CAR-T cell therapy, T cells are harvested from a patient's blood, engineered ex vivo to express CARs containing both antigen-binding and T cell-activating domains, expanded into a larger population, and administered to the patient. The CAR-T cells act as a living drug, binding to cancer cells and bringing about their destruction. When successful, the effects of CAR-T cell treatment tend to be long lasting, as evidenced by detection of CAR-T cell persistence and expansion in the patients long after clinical remission.

### 2. CAR Structure and Function

The antigen-binding domain of a CAR is an extracellular region that targets a surface antigen on tumor cells. Appropriate target antigens can be proteins, phosphorylated proteins, peptide-MHC, carbohydrates, or glycolipid molecules. Ideal target antigens are widely expressed on tumor cells to enable targeting of a high percentage of the cancer cells. Ideal candidate target antigens are also minimally expressed on normal tissues, limiting off-tumor, on-target toxicity. The antigen-binding domain of a CAR comprises a targeting moiety, such as an antibody single chain variable fragment (scFv), which is directed against the target antigen.

The T cell-activating domain of a CAR is intracellular and activates the T cell in response to the antigen-binding domain interacting with its target antigen. A T cell activating domain can contain one or more co-stimulatory domains, which are the intracellular domains of known activating T cell receptors. The selection and positioning of costimulatory domains within a CAR construct influence CAR-T cell function and fate, as costimulatory domains have differential impacts on CAR-T cell kinetics, cytotoxic function, and safety profile.

The extracellular antigen-binding and intracellular T cell-activating domains of CARs are linked by a transmembrane domain, hinge, and optionally, a spacer region. The hinge domain is a short peptide fragment that provides conformational freedom to facilitate binding to the target antigen on the tumor cell. It may be used alone or in conjunction with a spacer domain that projects the scFv away from the T cell surface. The optimal length of the spacer depends on the proximity of the binding epitope to the cell surface.

CAR-T therapy against the B-lymphocyte antigen CD19 (Kymriah®, Novartis) has shown promise in pediatric acute lymphocytic leukemia, and CAR-T therapy against B-cell maturation antigen (“bb2121,” a Celgene® and Bluebirdbio® collaboration) has shown promise against relapsed/refractory multiple myeloma. More recent data suggest that the CAR approach can be efficacious against solid tumors. A GD2 CAR natural killer T cell (NKT) therapy has shown activity in neuroblastoma (Heczey A, et al. Invariant NKT cells with chimeric antigen receptor provide a novel platform for safe and effective cancer immunotherapy. Blood; 124(18):2824-33, 2014), and mesothelin CAR-T with pembrolizumab has demonstrated anti-tumor activity in mesothelioma. However, additional targets for treating solid tumors are needed.

### 3. Challenges of CAR-T Cell Therapy

Unfortunately, the complexities of CAR-T cell-based therapy can lead to undesirable and unsafe effects. Toxic effects such as neurotoxicity and acute respiratory distress syndrome are potential adverse effects of CAR-T cell therapy and are potentially fatal. Cytokine release syndrome (CRS) is the most common acute toxicity associated with CAR-T cells. CRS occurs when lymphocytes are highly activated and release excessive amounts of inflammatory cytokines. Serum elevations of interleukin 2, interleukin 6, interleukin 1 beta, GM-CSF, and/or C-reactive protein are sometimes observed in patients with CRS when these factors are assayed. CRS is graded in severity and is diagnosed as one of grades 1-4 (mild to severe), with more serious cases clinically characterized by high fever, hypotension, hypoxia, and/or multi-organ toxicity in the patient. One study reported that 92% of acute lymphocytic leukemia patients treated with an anti-CD19 CAR-T cell therapy experienced CRS, and 50% of these patients developed grade 3-4 symptoms (Fitzgerald et al., Crit Care Med. 45(2): e124—e131 (2017)).

Another challenge to successful CAR-T cell immunotherapy is immunosuppression caused by characteristics of the tumor microenvironment (TME) of solid tumors. For example, Transforming Growth Factor beta (TGF-β) is a pleiotropic cytokine produced by many cell types in the liver (e.g., liver sinusoidal endothelial cells, Küpffer cells, intrahepatic natural killer (NK) cells, etc.) and in large amounts within cancer microenvironments (Dahmani et al., TGF-β in T Cell Biology: Implications for Cancer Immunotherapy. Cancers 2018, 10, 194, 1-21). TGF-β binds to TGFβR2, which recruits and phosphorylates TGFβR1, which once phosphorylated, in turn phosphorylates receptor-regulated SMADs (R-SMADs). The phosphorylated SMADs complex with coSMADs translocate to the nucleus to help regulate gene expression. In the context of T cells, TGF-β signaling suppresses CAR-T cell therapy effectiveness by inhibition of T cell proliferation, activation, and effector functions and by favoring regulatory T-cell differentiation. Therefore, TGF-β-associated immunosuppression is a significant hurdle that must be overcome to obtain effective and persistent CAR-T cell therapy for solid tumors.

### 4. Armoring

A recent approach to making CAR-T cells that are more resistant to tumor-associated immunosuppression is called “armoring.” Armoring is the molecular manipulation of a CAR-T cell to express one or more “armoring molecules” that can counter immunosuppression. For example, investigators recently reported modifying CAR-T cells to secrete PD-1-blocking single-chain variable fragments (scFv), which improved CAR-T cell anti-tumor activity in mouse models of PD-L1⁺ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). Others studies have demonstrated the effectiveness of armoring T cells with a dominant-negative TGF-β receptor type 2 (TGFβRIIDN) armoring molecule to neutralize the suppressive effects of TGF-β on T cells (Bollard et al., Tumor-Specific T-Cells Engineered to Overcome Tumor Immune Evasion Induce Clinical Responses in Patients With Relapsed Hodgkin Lymphoma, J Clin Oncol 36(11):1128-1139 (2018)). Currently, at least one clinical study is investigating the effectiveness of armoring anti-PSMA-CAR-T cells with a TGFβRIIDN armoring molecule for treating castrate-resistant prostate cancer (NCT03089203).

Therefore, additional CAR-T cell therapies are needed to augment the armamentarium of effective cancer treatments. Such therapies should include CAR-T cells that effectively treat cancer while minimizing the risk of developing dangerous inflammatory responses, such as CRS. In addition, such therapies should include CAR-T cells that can persist in the immunosuppressive TME of solid tumors.

### BRIEF SUMMARY OF THE INVENTION

This disclosure describes compositions and methods for using CAR-T cells to treat cancer. As described below, in a first aspect, an isolated nucleic acid sequence encoding (a) a chimeric antigen receptor (CAR), wherein the CAR comprises an antigen-binding domain specific for a cell surface antigen; and (b) an armoring molecule, wherein the armoring molecule counters immunosuppression of a cell in a tumor microenvironment when expressed on a surface of the cell.

In another aspect, the disclosure describes a cell comprising a nucleic acid sequence encoding a chimeric antigen receptor (CAR), and a TGFβRIIDN armoring molecule expressed on a surface of the cell.

In a further aspect, the disclosure describes a cell, comprising: an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45; and a TGFβRIIDN armoring molecule.

In yet another aspect, the disclosure describes a method of treating cancer, comprising: administering to a subject in need thereof a cell, wherein the cell comprises (a) an chimeric antigen receptor (CAR) specific for a cell surface antigen, and (b) an armoring molecule, wherein the armoring molecule counters immunosuppression of the cell in a tumor microenvironment of the cancer.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure. The drawings illustrate one or more embodiment(s) of the disclosure and together with the description serve to explain the principles and operation of the disclosure.

FIGS. 1A-1D. TGFβ1 and TGFβ gene signature and HCC development and survival. 1A. Fold change (loge) of TGFβ1 gene expression in normal solid tissue (left) and primary solid tumor of hepatocellular carcinoma (HCC, right). 1B. Survival curves (OS=overall survival) of TGFβ1 high expression versus TGFβ1 low expression in HCC. 1C. Fold change (loge) of TGFβ signaling in normal solid tissue (left) and solid tumor tissue (right). 1D. Survival curves of TGFβ high signaling versus low signaling.

FIGS. 2A and 2B. Human HCC samples are positive for TGFβ and TGFβ signaling. Semi-quantitative pathological assessment of (2A) TGF-b1 and (2B) phosphorylated SMAD2 (pSMAD2) in FFPE normal liver and HCC specimens. Each circle represents the data from one surgical resection. IHC intensity was define as: score 0, negative staining; score 1, minimal staining; score 2, moderate staining; score 3, strong staining.

FIGS. 3A and 3B. Armoring of GPC3 CAR-T with TGFβRIIDN. As contemplated herein, it is believed that armoring of GPC3 CAR-T cells with TGFβRIIDN will confer resistance to TGFβ, resulting in improved CAR-T effector function and tumor control. 3A. A schematic of TGF-β-mediated immunosuppression of unarmored CAR-T cells (FIG. modified from: Arrese et al, Current Protein & Peptide Science (2018) 19: 1172) 3B. A diagram of an armored CAR-T cell expressing a GPC3 CAR and a TGFβRIIDN armoring molecule. In some embodiments, a CAR and an armoring molecule can be fused, or separated from the CAR by a spacer peptide, at the C terminus.

FIG. 4. Expression of TGFβRIIDN. Flow cytometric analysis of TGFβRII and CAR on the surface of unarmored and armored CAR-T cells.

FIG. 5. TGFβRIIDN suppresses TGF-β signaling in CAR-T cells. 4B. Western blot analysis of phosphorylated SMAD2/3 and total SMAD 2/3 in untransduced (UT); unarmored, and armored (TGFβRIIDN) CAR-T cells after 0-45 minutes of rhTGFβ (1 ng/ml) exposure. β-Actin served as a positive loading control for all samples.

FIGS. 6A and 6B. Expression of TGFβRIIDN prevents TGF-β-induced suppression of effector cytokine transcription. (6A) IL2 and (6B) IFNG mRNA levels in purified TGFβRIIDN and unarmored GPC3 CAR-T cells stimulated for 6 hours with indicated concentration of plate-bound recombinant GPC3 and TGF-β1. Data are pooled from two independent experiments.

FIGS. 7A and 7B. TGFβRIIDN reduces TGF-β-mediated differentiation of GPC3 CAR-T cells to T_{RM} phenotype. 7A. Flow cytometric analysis of TGF-β-mediated differentiation of GPC3 CAR-T cells to tissue resident memory (T_{RM}) cells in the absence (left scatter plots) and presence (right scatter plots) of TGF-β in unarmored (upper scatter plots) and armored (lower scatter plots) GPC3 CAR-T cells. Y-axis=CD103 expression; X-axis=CAR expression. 7B. Percent of T_{RM} cell differentiation as evidenced by CD69+/CD103+ co-expression in unarmored (GPC3) and armored (GPC3 TGFβRIIDN) CAR-T cells treated with TGF-β.

FIGS. 8A, 8B, and 8C. Exploratory in vitro readouts in unarmored and armored CAR-T cells. 8A. Bright field images of real-time monitoring of CAR-T-mediated cytotoxicity during co-culture with GPC3+ tumor cells. Expansion of CAR-T cells was observed visually while in co-culture with GPC3+ tumor cells. 8B. CAR-T-mediated cytotoxicity during co-culture with GPC3+ tumor cells evaluated by xCelligence® real time impedance-based killing assay (RTCA) and expressed as Cell Index. 8C. Quantification of tumor cell (HEP3B or HUH-7 cells)-induced CAR-T cell proliferation in unarmored and armored GPC3 CAR-T cells (AZ: in-house vector backbone; LN: Lentigen vector backbone) in the presence or absence of TGF-β. TGF-β suppresses tumor GPC3-induced proliferation of CAR-T cells after 4 days in co-culture. Armored GPC3 CAR-T cells were not susceptible to suppression.

FIG. 9. TGFβRIIDN-armored CAR-T cells and in vivo reduction of tumor volume in xenograft model. Huh7-TGF-β over-expressing xenograft model. Untransduced T cells, unarmored or TGFβRIIDN-armored GPC3 CAR-T cells were infused in mice bearing Huh7 tumors engineered to over-express TGF-β. Tumor volume was measured by weekly (10 mice/group).

FIG. 10. Increased number of tumor-infiltrating lymphocytes in mice treated with TGFβRIIDN-armored CAR-T cells. Mice bearing Huh7-TGF-β over-expressing tumors were dosed with 7×10⁶ untransduced T cells, unarmored or TGFβRIIDN-armored GPC3 CAR-T. 10A. Number of CARP cells in the tumor of mice harvested at the indicated time points. 10B. Representative example of FACS data summarized in 10A.

FIG. 11. Increased number of TGFβRIIDN-armored CAR-T cells in the spleen. Mice bearing Huh7-TGF-β over-expressing tumors were dosed with 7×10⁶ untransduced T cells, unarmored or TGFβRIIDN-armored GPC3 CAR-T cells. 11A. Number of CARP cells in the spleen of mice harvested at the indicated time points. 11B. Representative example of FACS data summarized in 11A.

FIG. 12. Expression of TGFβRII on the surface of CARP cells ex vivo. 12A. Expression of CAR and TGFβRII on the surface of lymphocytes infiltrating the spleen or the tumor 14 days after the infusion in mice bearing Huh7-TGF-β over-expressing tumors. (FMO: fluorescence minus one) 12B. Mean Fluorescence Intensity (MFI) of TGFβRII on the surface of CAR⁺ or CAR⁻ CD8⁺ T cells in the tumor.

FIG. 13. Decreased PD1 and LAG3 expression on the surface of TGFβRIIDN-armored CAR-T cells in the tumor. Expression of PD1 (A) and LAG3 (B) on the surface of tumor-infiltrating CD8⁺ and CD4⁺CAR⁺ T cells 14 days after the infusion in mice bearing Huh7-TGF-β over-expressing tumors.

FIG. 14. Decreased expression of CD70 and increased expression of CD27 cells on the surface of TGFβRIIDN-armored CAR-T cells in the tumor. Frequency of CD70⁺CD27⁻ (A) and of CD70⁺CD27⁻ (B) on the surface of tumor-infiltrating CD8⁺ and CD4⁺CAR⁺ T cells 14 days after the infusion in mice bearing Huh7-TGF-β over-expressing tumors.

FIG. 15. Immunophenotype of CAR-T cells in the spleen. Expression of PD1 (15A), LAG3 (15B), and CD27/CD70 (15C) on the surface of CD8⁺ and CD4⁺CAR⁺ T cells in the spleen 14 days after the infusion in mice bearing Huh7-TGF-β over-expressing tumors.

FIG. 16. Analysis of serum IFN-γ. Concentration of IFN-γ detected in the serum of mice bearing Huh7-TGF-β over-expressing tumors. and infused with untransduced T cells, unarmored or armored GPC3 CAR-T cells (Baseline A: before tumor implantantion; Baseline B: before CAR-T infusion). FIG. 17. Analysis of serum AFP. Concentration of AFP detected in the serum of mice bearing Huh7-TGF-β over-expressing tumors and infused with untransduced T cells, unarmored or armored GPC3 CAR-T cells (Baseline A: before tumor implantantion; Baseline B: before CAR-T infusion).

FIG. 18. TGFβRIIDN-armored CAR-T cells and in vivo reduction of tumor volume in TGFβ(−) PDX models. A-C represent the tumor volume of three different GPC3⁺ TGF-β⁻ Hepatocellular Carcinoma (HCC) patient derived xenograft (PDX) models. For each model, 5×10⁶ untransduced T cells, unarmored or armored GPC3 CAR-T cells were infused in tumor-bearing mice, and tumor volume was measured bi-weekly (5 mice/group).

FIG. 19. TGFβRIIDN-armored CAR-T cells and in vivo reduction of tumor volume in TGFβ(+) PDX models. A-E represent the tumor volume of five different GPC3⁺ TGF-β⁺ Hepatocellular Carcinoma (HCC) patient derived xenograft (PDX) models. For each model 5×10⁶ untransduced T cells, unarmored or armored GPC3 CAR-T cells were infused in tumor bearing mice, and tumor volume was measured bi-weekly (5 mice/group).

### DETAILED DESCRIPTION

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Unless otherwise clear from context, all numerical values provided herein are modified by the term “about.”

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “polypeptide” refers to a molecule composed of monomers (amino acids) linearly linked by amide bonds (also known as peptide bonds). The term “polypeptide” refers to any chain or chains of two or more amino acids. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

An “isolated” substance, e.g., isolated nucleic acid, is a substance that is not in its natural milieu, though it is not necessarily purified. For example, an isolated nucleic acid is a nucleic acid that is not produced or situated in its native or natural environment, such as a cell. An isolated substance can have been separated, fractionated, or at least partially purified by any suitable technique.

As used herein, the terms “antibody” and “antigen-binding fragment thereof” refer to at least the minimal portion of an antibody which is capable of binding to a specified antigen which the antibody targets, e.g., at least some of the complementarity determining regions (CDRs) of the variable domain of a heavy chain (VH) and the variable domain of a light chain (VL) in the context of a typical antibody produced by a B cell. Antibodies or antigen-binding fragments thereof can be or be derived from polyclonal, monoclonal, human, humanized, or chimeric antibodies, single chain antibodies, epitope-binding fragments, e.g., Fab, Fab′ and F(ab′)2, Fd, Fvs, single-chain Fvs (scFvs), single-chain antibodies, disulfide-linked Fvs (sdFvs), fragments comprising either a VL or VH domain alone or in conjunction with a portion of the opposite domain (e.g., a whole VL domain and a partial VH domain with one, two, or three CDRs), and fragments produced by a Fab expression library. ScFv molecules are known in the art and are described, e.g., in U.S. Pat. No. 5,892,019. Antibody molecules encompassed by this disclosure can be of or be derived from any type (e.g., IgG, IgE, IgM, IgD, IgA, and IgY), class (e.g., IgG1, IgG2, IgG3, IgG4, IgA1 and IgA2), or subclass of immunoglobulin molecule. The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, MD. (1991).

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA.

As used herein, the term “vector” can refer to a nucleic acid molecule as introduced into a host cell, thereby producing a transformed host cell. A vector can include nucleic acid sequences that permits it to replicate in a host cell, such as an origin of replication. A vector can also include one or more selectable marker gene and other genetic elements known in the art. Specific types of vector envisioned here can be associated with or incorporated into viruses to facilitate cell transformation.

A “transformed” cell, or a “host” cell, is a cell into which a nucleic acid molecule has been introduced by molecular biology techniques. All techniques by which a nucleic acid molecule can be introduced into such a cell, including transfection with viral vectors, transformation with plasmid vectors, and introduction of naked DNA by electroporation, lipofection, and particle gun acceleration are contemplated herein.

As used herein, the term “affinity” refers to a measure of the strength of the binding of an antigen or target (such as an epitope) to its cognate binding domain (such as a paratope). As used herein, the term “avidity” refers to the overall stability of the complex between a population of epitopes and paratopes (i.e., antigens and antigen binding domains).

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the term an “effective amount” or a “therapeutically effective amount” of an administered therapeutic substance, such as a CAR-T cell, is an amount sufficient to carry out a specifically stated or intended purpose, such as treating or treatment of cancer. An “effective amount” can be determined empirically in a routine manner in relation to the stated purpose.

### 2. Overview

The present disclosure is directed to compositions and methods for treating cancer using chimeric antigen receptor (CAR) cell therapy. More particularly, the present disclosure concerns CAR cell therapies in which the transformed cells, such as T cells, express CARs that for example target Glypican-3 (GPC3). The CAR constructs transformed cells expressing the constructs, and the therapies utilizing the transformed cells disclosed herein can provide robust cancer treatments with minimized risk of cytokine release syndrome (CRS) or indiscriminate cytokine release in non-GPC3 expressing cells.

Without wishing to be bound by theory, GPC3 is believed to be a viable cancer target across multiple modalities, including bispecific T cell engagers, CAR cells, as well as monoclonal antibodies and antibody-drug conjugates (ADCs). GPC3, an onco-fetal antigen, is a GPI-linked heparin sulfate proteoglycan. GPC3 stabilizes the Wnt-Fzd interaction, stimulating Wnt signaling. GPC3 competes with Patched for Hh binding, relieving Smoothened inhibition, and inducing GPC3 degradation. Both pathways have been shown to stimulate hepatocellular carcinoma (HCC) growth. And, GPC3 expression levels have been shown to correlate with stage and grade of HCC.

Further, it is believed that GPC3 is a promising target for CAR cell therapy. Therefore, antibodies and CAR constructs derivized from these antibodies have been developed as described herein.

An additional aspect of the present disclosure includes CAR-T cells, such as those targeting GPC3 and others that are armored with TGFβRIIDN to protect the CAR-T cells against TGF-O-associated immunosuppression for example, of solid tumors.

### 3. CAR Construct Design

CAR constructs of the present disclosure can have several components, many of which can be selected based upon a desired or refined function of the resultant CAR construct. In addition to an antigen-binding domain, CAR constructs can have a spacer domain, a hinge domain, a signal peptide domain, a transmembrane domain, and one or more costimulatory domains. Selection of one component over another (i.e., selection of a specific co-stimulatory domain from one receptor versus a co-stimulatory domain from a different receptor) can influence clinical efficacy and safety profiles.

### 4. Antigen Binding Domain

Antigen binding domains contemplated herein can include antibodies or one or more antigen-binding fragments thereof. One contemplated CAR construct targeting GPC3 comprises a single chain variable fragment (scFv) containing light and heavy chain variable regions from one or more antibodies specific for GPC3 that are either directly linked together or linked together via a flexible linker (e.g., a repeat of GGGGS having 1, 2, 3 or more repeats (SEQ ID NO: 48)).

The antigen-binding domain of a CAR as disclosed herein can vary in its binding affinity for the target protein. The relationship between binding affinity and efficacy can be more nuanced in the context of CARs as compared with antibodies, for which higher affinity is typically desirable. For example, preclinical studies on a receptor tyrosine kinase-like orphan receptor 1 (ROR1)-CAR derived from a high-affinity scFv (with a dissociation constant of 0.56 nM) resulted in an increased therapeutic index when compared with a lower-affinity variant. Conversely, other examples have been reported that engineering the scFv for lower affinity improves the discrimination among cells with varying antigen density. This could be useful for improving the therapeutic specificity for antigens differentially expressed on tumor versus normal tissues.

A variety of methods can be used to ascertain the binding affinity of the antigen-binding domain. In some embodiments, methodologies that exclude avidity effects can be used. Avidity effects involve multiple antigen-binding sites simultaneously interacting with multiple target epitopes, often in multimerized structures. Thus, avidity functionally represents the accumulated strength of multiple interactions. An example of a methodology that excludes avidity effects is any approach in which one or both of the interacting proteins is monomeric/monovalent since multiple simultaneous interactions are not possible if one or both partners contain only a single interaction site.

### 5. Spacer Domain

A CAR construct of the present disclosure can have a spacer domain to provide conformational freedom to facilitate binding to the target antigen on the target cell. The optimal length of a spacer domain may depend on the proximity of the binding epitope to the target cell surface. For example, proximal epitopes can require longer spacers and distal epitopes can require shorter ones. Besides promoting binding of the CAR to the target antigen, achieving an optimal distance between a CAR cell and a cancer cell may also help to occlude sterically large inhibitory molecules from the immunological synapse formed between the CAR cell and the target cancer cell. A CAR can have a long spacer, an intermediate spacer, or a shorter spacer. Long spacers can include a CH2CH3 domain (˜220 amino acids) of immunoglobulin G1 (IgG1) or IgG4 (either native or with modifications common in therapeutic antibodies, such as a S228P mutation), whereas the CH3 region can be used on its own to construct an intermediate spacer (˜120 amino acids). Shorter spacers can be derived from segments (<60 amino acids) of CD28, CD8α, CD3 or CD4. Short spacers can also be derived from the hinge regions of IgG molecules. These hinge regions may be derived from any IgG isotype and may or may not contain mutations common in therapeutic antibodies such as the S228P mutation mentioned above.

### 6. Hinge Domain

A CAR can also have a hinge domain. The flexible hinge domain is a short peptide fragment that provides conformational freedom to facilitate binding to the target antigen on the tumor cell. It may be used alone or in conjunction with a spacer sequence. The terms “hinge” and “spacer” are often used interchangeably—for example, IgG4 sequences can be considered both “hinge” and “spacer” sequences (i.e., hinge/spacer sequences).

A CAR can further include a sequence comprising a signal peptide. Signal peptides function to prompt a cell to translocate the CAR to the cellular membrane. Examples include an IgG1 heavy chain signal polypeptide, Ig kappa or lambda light chain signal peptides, granulocyte-macrophage colony stimulating factor receptor 2 (GM-CSFR2 or CSFR2) signal peptide, a CD8a signal polypeptide, or a CD33 signal peptide.

### 7. Transmembrane Domain

A CAR can further include a sequence comprising a transmembrane domain. The transmembrane domain can include a hydrophobic a helix that spans the cell membrane. The properties of the transmembrane domain have not been as meticulously studied as other aspects of CAR constructs, but they can potentially affect CAR expression and association with endogenous membrane proteins. Transmembrane domains can be derived, for example, from CD4, CD8α, or CD28.

### 8. Costimulatory Domain

A CAR can further include one or more sequences that form a co-stimulatory domain. A co-stimulatory domain is a domain capable of potentiating or modulating the response of immune effector cells. Co-stimulatory domains can include sequences, for example, from one or more of CD3zeta (or CD3z), CD28, 4-1BB, OX-40, ICOS, CD27, GITR, CD2, IL-2Rβ and MyD88/CD40. The choice of co-stimulatory domain influences the phenotype and metabolic signature of CAR cells. For example, CD28 co-stimulation yields a potent, yet short-lived, effector-like phenotype, with high levels of cytolytic capacity, interleukin-2 (IL-2) secretion, and glycolysis. By contrast, T cells modified with CARs bearing 4-1BB costimulatory domains tend to expand and persist longer in vivo, have increased oxidative metabolism, are less prone to exhaustion, and have an increased capacity to generate central memory T cells.

### 9. Cells

CAR-based cell therapies can be used with a variety of cell types, such as lymphocytes. Particular types of cells that can be used include T cells, Natural Killer (NK) cells, Natural Killer T (NKT) cells, Invariant Natural Killer T (iNKT) cells, alpha beta T cells, gamma delta T cells, viral-specific T (VST) cells, cytotoxic T lymphocytes (CTLs), and regulatory T cells (Tregs). In one embodiment, CAR cells for treating a subject are autologous. In other embodiments, the CAR cells may be from a genetically similar, but non-identical donor (allogeneic).

### 10. CAR Cell Production

CAR constructs of the present disclosure can include some combination of the modular components described herein. For example, in some embodiments of the present disclosure, a CAR construct comprises a GPC3 scFv antigen-binding domain. In some embodiments, a CAR comprises a GPC3-2 scFv antigen-binding domain. In some embodiments of the present disclosure, a CAR construct comprises a CSFR2 signal peptide. In some embodiments, a CAR construct comprises an IgG4P hinge/spacer domain carrying an S228P mutation. In some embodiments, a CAR construct comprises a CD28 transmembrane.

Different co-stimulatory domains can be utilized is the CAR constructs of the present disclosure. In some embodiments, a CAR construct comprises a co-stimulatory domain from the intracellular domain of CD3z. In some embodiments, a CAR construct comprises a CD28 co-stimulatory domain. In some embodiments, a CAR construct comprises a 4-1BB co-stimulatory domain. In some embodiments, a CAR construct comprises co-stimulatory domains from CD3z and CD28. In some embodiments, a CAR construct comprises co-stimulatory domains from CD3z and 4-1BB. In some embodiments, a CAR construct comprises co-stimulatory domains from all of CD3z, CD28, and 4-1BB. In some embodiments, a CAR construct comprises co-stimulatory domains from ICOS, OX-40, and/or GITR.

### 11. CAR Construct Assessment

Constructs of the present disclosure were compared and assessed based on safety as well as persistence and establishment of central memory. The lower affinity (high off-rate) scFv, GPC3, was assessed favorably on account of its improved safety. The 4-1BB and CD3z co-stimulatory domains (both in the same construct) were assessed favorably based on their contribution to improved persistence and favorable in vivo phenotype (more central memory).

### 12. CAR Embodiments

In some embodiments, the present disclosure provides an isolated nucleic acid sequence encoding a chimeric antigen receptor (CAR) that comprises an antigen-binding domain specific for a surface antigen on a tumor cell. In some embodiments, the cell surface antigen is a protein, a phosphorylated protein, a peptide-MHC, a carbohydrate, or a glycolipid molecule.

Examples of contemplated cell surface antigens include CD10, CD16, CD19, CD20, CD22, CD123, CD30, CD34, CD47, CD56, CD80, CD86, CD117, CD133, CD138, CD171, CD37, CD38, CD5, CD7, CD79, 5T4, AFP, AXL, BCMA, B7H3, CDH3, CDH6, CLDN6, CLDN18, CLL-1, CMV, CS1, DLL3, DR5, FBP, GD2, GFRA1, GPA33, GPC3, IL-1-RAP, IL17RA, ITGB7, EBV, ERBB1/EGFR, ERBB2/Her-2, ERBB3, ERBB4, cMet, EGFR vIII, FAP, FOLR1, CEA, CEACAM6, EphA2, HSV-1, HSV-2, HTLV, HPV16-E6, HPV16-E7, IL13Ra2, Igκ chain, LGR5, LMP1, LeY, LRP8, MG7, MR1, NRCAM, PMEL, NKG2D ligand, PRAME, PRLR, PVR, ROR1, ROR2, SSX2, STEAP1, STEAP2, TACI, TIM3, TRBC1, VEGFR-2, EPCAM1, VCAM1, VIPR2, MAGE-A1, MAGE-A3, MAGE-A4, mesothelin (MSLN), MUC1, MUC16, NY-ESO-1, WT1, PDL1, CAIX, CD70, PSMA, and PSCA. Other cell surface antigens are also contemplated herein.

In some embodiments, the present disclosure provides an isolated nucleic acid sequence encoding a chimeric antigen receptor (CAR) that comprises an antigen binding domain specific for glypican 3 (GPC3). The antigen binding domain has an equilibrium dissociation constant (K_{D}) of about 100 nanomolar (nM) or less, and the CAR construct does not induce cytokine production in GPC3-cells. In some embodiments, the antigen-binding domain includes an antibody or antigen-binding fragment thereof. The antigen-binding domain can be a Fab or a single chain variable fragment (scFv). In some embodiments, the antigen-binding domain is an scFv comprising the nucleic acid sequence of SEQ ID NO: 33 or SEQ ID NO: 34.

In some embodiments, the CAR further includes a transmembrane domain, a costimulatory domain, and a signal domain. The transmembrane domain can be a CD28 transmembrane domain. The costimulatory domain can be one or more of CD3zeta (or CD3z), CD28, 4-1BB, OX-40, ICOS, CD27, GITR, CD2, IL-2RP and MyD88/CD40costimulatory domains. In one specific embodiment, the costimulatory domain is one or more of CD28, 4-1BB, and CD3zeta costimulatory domains. The signal domain can be a sequence encoding a CSFR2 signal peptide.

In some embodiments, the isolated nucleic acid sequence can include a hinge/spacer domain. The hinge/spacer domain can be an IgG4P hinge/spacer.

In some specific embodiments, an isolated nucleic acid sequence encoding a chimeric antigen receptor (CAR) can have the sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 26.

In other embodiments, the present disclosure provides an anti-GPC3 chimeric antigen receptor (CAR) including an antigen-binding domain. The antigen-binding domain can be an antibody, Fab, or scFv comprising a heavy chain variable region (VH) and a light chain variable region (VL). In some embodiments, the VH can have a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39. In some embodiments, the VL can have a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments, the VH can be the amino acid sequence of SEQ ID NO: 27 or SEQ ID NO: 29, and the VL can be the amino acid sequence of SEQ ID NO: 28 or SEQ ID NO: 30. In some embodiments, the CAR further can have a transmembrane domain, a costimulatory domain, and a signal domain.

In some specific embodiments, the anti-GPC3 CAR can have the amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

In other embodiments, the present disclosure provides a vector comprising a nucleic acid sequence encoding a chimeric antigen receptor (CAR). The nucleic acid sequence can be SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In other embodiments, the present disclosure provides a cell comprising a vector having a nucleic acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In other embodiments, the present disclosure provides a cell having a nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, and wherein the CAR construct does not induce cytokine production in GPC3-cells. For example, the nucleic acid sequence can be SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 26, SEQ ID NO: 33, or SEQ ID NO: 34.

In other embodiments, the present disclosure provides a cell expressing an anti-GPC3 chimeric antigen receptor (CAR) on an extracellular surface thereof. The CAR can have an antigen-binding domain that can be an antibody, a Fab, or an scFv each having a heavy chain variable region (VH) and a light chain variable region (VL). The VH can include a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39. The VL can include a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45.

In some embodiments, the VH can have the amino acid sequence of SEQ ID NO: 27 or SEQ ID NO: 29. In some embodiments, the VL can have the amino acid sequence of SEQ ID NO: 28 or SEQ ID NO: 30. The CAR can further include a transmembrane domain, a costimulatory domain, and a signal domain. The cell express a CAR having an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25.

In some embodiments, the present disclosure provides a T cell, a Natural Killer (NK) cell, a cytotoxic T lymphocyte (CTL), and/or a regulatory T cell that express a CAR on an extracellular surface thereof, and the CAR can have an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 25. Such cells can exhibit an anti-tumor immunity upon contacting a tumor cell expressing GPC3.

### 13. Treatment of Cancers with CARs

In some embodiments, the present disclosure provides CAR cells for treatment of cancer. The compositions (e.g., antibodies, CAR constructs, and CAR cells) and methods of their use described herein are especially useful for inhibiting neoplastic cell growth or spread. In some aspects, they are particularly useful for inhibiting neoplastic cell growth in which GPC3 plays a role.

Neoplasms treatable by the compositions of the disclosure include solid tumors, for example, those of the liver, lung, or ovary. However, the cancers listed herein are not intended to be limiting. For example, types of cancer that are contemplated for treatment herein include, for example, NSCLC, advanced solid malignancies, biliary tract neoplasms, bladder cancer, colorectal cancer, diffuse large b-cell lymphoma, esophageal neoplasms, esophageal squamous cell carcinoma, extensive stage small cell lung cancer, gastric adenocarcinoma, gastric cancer, gastroesophageal junction cancer, head and neck cancer, head and neck squamous cell carcinoma, hepatocellular carcinoma, Hodgkin lymphoma, lung cancer, melanoma, mesothelioma, metastatic clear cell renal carcinoma, metastatic melanoma, metastatic non-cutaneous melanoma, multiple myeloma, nasopharyngeal neoplasms, non-Hodgkin lymphoma, ovarian cancer, fallopian tube cancer, peritoneal neoplasms, pleural mesothelioma, prostatic neoplasms, recurrent or metastatic PD-L1 positive or negative SCCHN, recurrent squamous cell lung cancer, renal cell cancer, renal cell carcinoma, SCCHN, hypo pharyngeal squamous cell carcinoma, laryngeal squamous cell carcinoma, small cell lung cancer, squamous cell carcinoma of the head and neck, squamous cell lung carcinoma, TNBC, transitional cell carcinoma, unresectable or metastatic melanoma, urothelial cancer, and urothelial carcinoma.

In one embodiment, cancers contemplated for treatment here include any that express GPC3 on the cell surfaces of the cancer cells. In one specific example, cancers contemplated for treatment herein include hepatocellular carcinoma, non-small cell lung cancer, ovarian cancer, and squamous cell lung carcinoma.

### 14. Armoring

In some embodiments, the present disclosure provides “armored” cells, such as CAR-T cells that have one or more genetic modifications that enhance or optimize cell function by protecting the cell against an environmental insult, such as an immunosuppressive cytokine or an immunosuppressive TME. Genetic modifications include, but are not limited to, enhanced secretion of cytokines, expression of ligands that interact with immune cells such as T cell, macrophages, and regulatory T cells, or an alteration of functional characteristics. One of skill in the art will understand that armoring a cell, such as a T cell, can provide many additional benefits not described herein that allow for T cell survival in the immunosuppressive TME.

In some embodiments, a cell can include a chimeric antigen receptor (CAR) comprising a tumor specific antigen binding domain, wherein the antigen binding domain comprises an antibody, Fab, or an scFv comprising a heavy chain variable region (VH) and a light chain variable region (VL); and a Transforming Growth Factor beta (TGF-β) receptor type 2 dominant negative (TGFβRIIDN) armoring molecule.

In some embodiments, an armored cell can include a nucleic acid sequence encoding a chimeric antigen receptor (CAR), wherein the CAR comprises an antigen binding domain specific for glypican 3 (GPC3), wherein the antigen binding domain has an equilibrium dissociation constant (K_{D}) of about 100 nanomolar (nM) or less, wherein the CAR construct does not induce cytokine production in GPC3-cells, and wherein the cell expresses a TGFβRIIDN armoring molecule.

In some embodiments, an armored cell can include an anti-GPC3 chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and wherein the VL comprises a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45; and a TGFβRIIDN armoring molecule.

### 15. Methods of Treatment

CAR-modified cells of the present invention, such as CAR-T cells, may be administered alone or as a pharmaceutical composition with a diluent and/or other components associated with cytokines or cell populations. Briefly, pharmaceutical compositions of the invention can include, for example, CAR-T cells as described herein, with one or more pharmaceutically or physiologically acceptable carrier, diluent, or excipient. Such compositions can comprise buffers such as neutral buffered saline, buffered saline, and the like; sulfates; carbohydrates such as glucose, mannose, sucrose, or dextrans, mannitol; proteins, polypeptides, or amino acids such as glycine; antioxidants; chelating agents such as EDTA or glutathione; adjuvants (e.g., aluminum hydroxide); and preservatives. The pharmaceutical compositions of the invention may be adapted to the treatment (or prophylaxis).

The CAR-modified cells can also be administered in conjunction with one or more additional therapies. In one embodiment, the additional therapies can include anti-cytokine antibodies. For example, one or more anti-TNFα antibodies can be used to attenuate toxicity and promote anti-tumor activity at higher CAR-T doses, which can be associated with CRS-like symptoms and weight loss.

The number of CAR cells administered per dose, the number of doses, and frequency of dosing will depend on various parameters such as the patient's age, weight, clinical assessment, tumor type, tumor burden, and/or other factors, including the judgment of the attending physician. Any acceptable route of administration is contemplated, such as, without limitation, administration intravenous (e.g., intravenous infusion), parenteral, or subcutaneous routes of administration.

In a particular embodiment, a treatment regimen contemplated can include one or more biological components, such as a CAR-T cell and an anticancer antibody and/or a chemotherapeutic component. For example, it is contemplated that a treatment regimen can additionally include an immune checkpoint inhibitor (ICI), such as those that target the PD-1/PD-L1 axis (PDX) and other immune-oncology (IO) treatments, such as immune system agonists.

Contemplated antibodies include an anti-PD-L1 antibody such as durvalumab (MEDI4736), avelumab, atezolizumab, KN035, an anti-PD-1 antibody such as nivolumab, pembrolizumab, cemiplimab, SHR1210, IB1I308, PDR001, Anti-PD-1, BGB-A317, BCD-100, and JS001, and an anti-CTLA4 antibody, such as tremelimumab or ipilimumab. Additional antibodies are also contemplated herein. Any therapeutically effective antibody subparts are also contemplated herein.

Information regarding durvalumab (or fragments thereof) for use in the methods provided herein can be found in U.S. Pat. Nos. 8,779,108; 9,493,565; and 10,400,039 the disclosures of which are incorporated herein by reference in their entirety. In a specific aspect, durvalumab or an antigen-binding fragment thereof for use in the methods provided herein comprises the variable heavy chain and variable light chain CDR sequences of the 2.14H90PT antibody as disclosed in the aforementioned U.S. patents.

Information regarding tremelimumab (or antigen-binding fragments thereof) for use in the methods provided herein can be found in U.S. Pat. No. 6,682,736 (in which tremelimumab is referred to as 11.2.1), the disclosure of which is incorporated herein by reference in its entirety.

Additional therapeutics (chemotherapies or biologics) contemplated herein include without limitation cisplatin/gemcitabine or methotrexate, vinblastine, ADRIAMYCIN™ (doxorubicin), cisplatin (MVAC), carboplatin-based regimen, or single-agent taxane or gemcitabine, temozolomide, or dacarbazine, vinflunine, docetaxel, paclitaxel, nab-paclitaxel, Vemurafenib, Erlotinib, Afatinib, Cetuximab, Bevacizumab, Erlotinib, Gefitinib, and/or Pemetrexed. Further examples include drugs targeting DNA damage repair systems, such as poly (ADP-ribose) polymerase 1 (PARP1) inhibitors and therapeutics inhibiting WEE1 protein kinase activity, ATR protein kinase activity, ATM protein kinase activity, Aurora B protein kinase activity, and DNA-PK activity.

Any therapeutic compositions or methods contemplated herein can be combined with one or more of any of the other therapeutic compositions and methods provided herein.

In some embodiments, the present disclosure provides a method of treating cancer including administering to a subject in need thereof an effective amount of a cell comprising an anti-GPC3 chimeric antigen receptor (CAR) comprising an antigen-binding domain and an armoring molecule that counters immunosuppression of the cell in a tumor microenvironment when expressed on a surface of the cell. In another aspect, the disclosure describes antigen-binding domain can be an antibody, Fab, or scFv comprising a heavy chain variable region (VH) and a light chain variable region (VL). The VH can include a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39. The VL can include a CDR1 comprising the amino acid sequence of SEQ ID NO: 40 or SEQ ID NO: 43, a CDR2 comprising the amino acid sequence of SEQ ID NO: 41 or SEQ ID NO: 44, and a CDR3 comprising the amino acid sequence of SEQ ID NO: 42 or SEQ ID NO: 45. In some embodiments, the method further inhibits tumor growth, induces tumor regression, and/or prolongs survival of the subject.

In some embodiments, the armoring molecule is TGFβRIIDN.

In some embodiments, the cell is an autologous cell. For example, the autologous cell can be selected from the group consisting of a T cell, a Natural Killer (NK) cell, a cytotoxic T lymphocyte (CTL), and a regulatory T cell.

In some embodiments, the cancer treated by the method is a solid tumor. For example, the cancer can be hepatocellular carcinoma, non-small cell lung cancer, ovarian cancer, and/or squamous cell lung carcinoma. In a specific embodiment, the cancer is hepatocellular carcinoma.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

### EXAMPLES

The Examples that follow are illustrative of specific embodiments of the disclosure, and various uses thereof. They are set forth for explanatory purposes only and should not be construed as limiting the scope of the disclosure in any way. A description of terms is provided in Table 1.

TABLE 1
Description of terms

| Term | Description |
|---|---|
| GPC3-1 | Anti-GPC3 scFv (lower affinity) |
| GPC3-2 | Anti-GPC3 scFv (higher affinity) |
| GPC3-4 | Prior art anti-GPC3 scFv |
| GPC3-3 | Prior art anti-GPC3 scFv |
| BZ | Intracellular domain of CAR with two co- |
| | stimulatory domains of 4-1BB and CD3zeta |
| TZ | Intracellular domain of CAR with a truncated |
| | CD3zeta signaling domain (acting as a signaling |
| | incompetent control) |
| 28Z | Intracellular domain of CAR with two co-stimulatory |
| | domains of CD28 and CD3zeta |
| 28BZ | Intracellular domain of CAR with three co-stimulatory |
| | domains of CD28, 4-1BB, and CD3zeta |
| TGFβRIIDN | Dominant-negative TGF-β receptor type 2 molecule |
| GPC3-1 BZ | CAR with GPC3 scFv, CSFR2 signal peptide, |
| | IgG4P hinge region, and both 4-1BB and CD3z |
| | co-stimulatory domains |
| GPC3-2 BZ | CAR with GPC3-2 scFv, CSFR2 signal peptide, |
| | IgG4P hinge region, and both 4-1BB and CD3z |
| | co-stimulatory domains |
| aCART-T | CAR-T cell with GPC3 scFv, CSFR2 signal peptide, |
| | IgG4P hinge region, and both 4-1BB and CD3z co- |
| | stimulatory domains and armored with TGFβRIIDN |
| CSFR2 | Signal peptide used in all CAR constructs |
| IgG4P | Hinge sequence used in all CAR constructs |
| Hep3B | Hepatocellular carcinoma model |
| HUH7 | Resistant hepatocellular carcinoma (HCC) model |

### Example 1: TGFβ Gene Expression and Signaling in Liver Hepatocellular Carcinoma Summary

In the present example, TGFβ 1 gene expression and TGFβ signaling were compared in normal liver versus liver hepatocellular carcinoma (LIHC).

### Methods

Data from TCGA cohort was used for this analysis. TGFβ 1 gene expression and TGFβ signaling signature in normal liver and tumor tissue from TCGA were compared using t-test. TGFβ signaling signature was formed from the mean expression levels of the following genes: TGFBR1, SMAD7, TGFB1, SMURF2, SMURF1, BMPR2, SKIL, SKI, ACVR1, PMEPA1, NCOR2, SERPINE1, JUNB, SMAD1, SMAD6, PPP1R15A, TGIF1, FURIN, SMAD3, FKBP1A, MAP3K7, BNMR1A, CTNNB1, HIPK2, KLF10, BMP2, ENG, APC, PPM1A, XIAP, CDH1, ID1, LEFTY2, CDKN1C, TRIM33, RAB31, TJP1, SLC20A1, CDK9, ID3, NOG, ARID4B, IFNGR2, ID2, PPP1CA, SPTBN1, WWTR1, BCAR3, THBS1, FNTA, HDAC1, UBE2D3, LTBP2, and RHOA,

Kaplan-Meier analyses of overall survival (OS) were conducted with LIHC data from TCGA. The data were grouped according to high (>66th) and low (<66th) TGFβ 1 gene expression and TGFβ signaling signature. The P values were determined using the log-rank test.

### Results

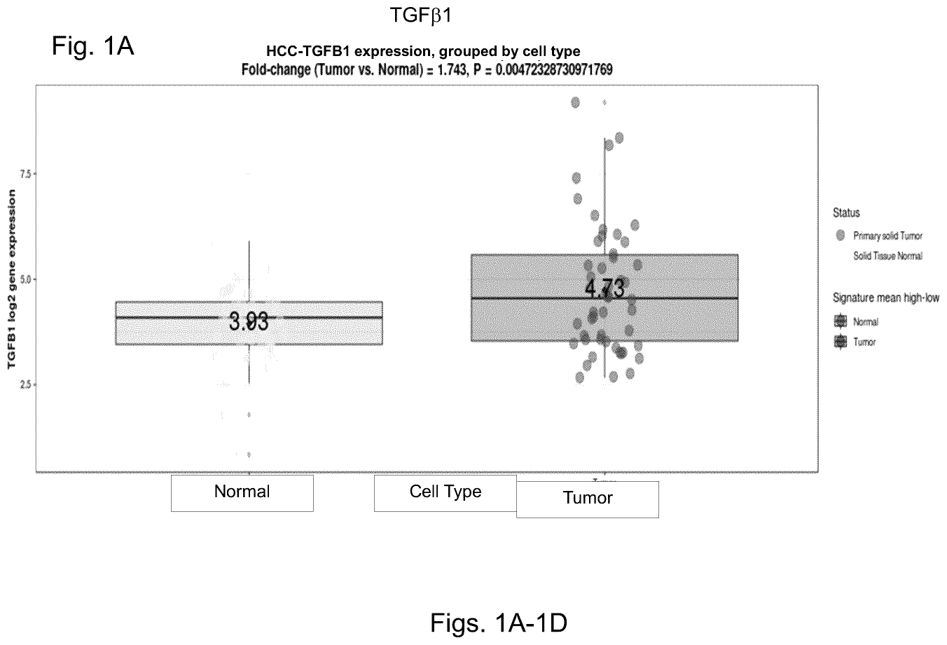

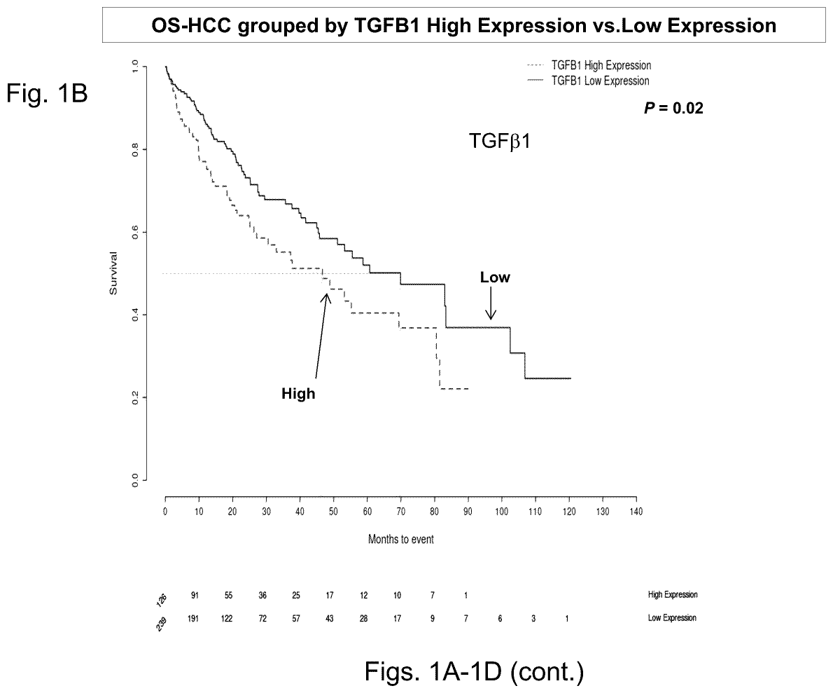

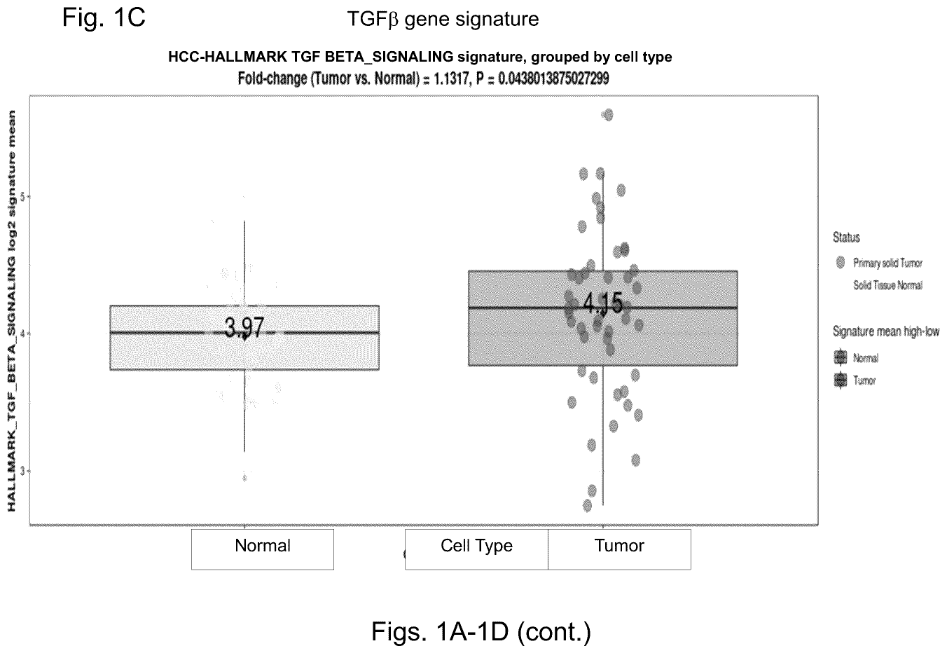

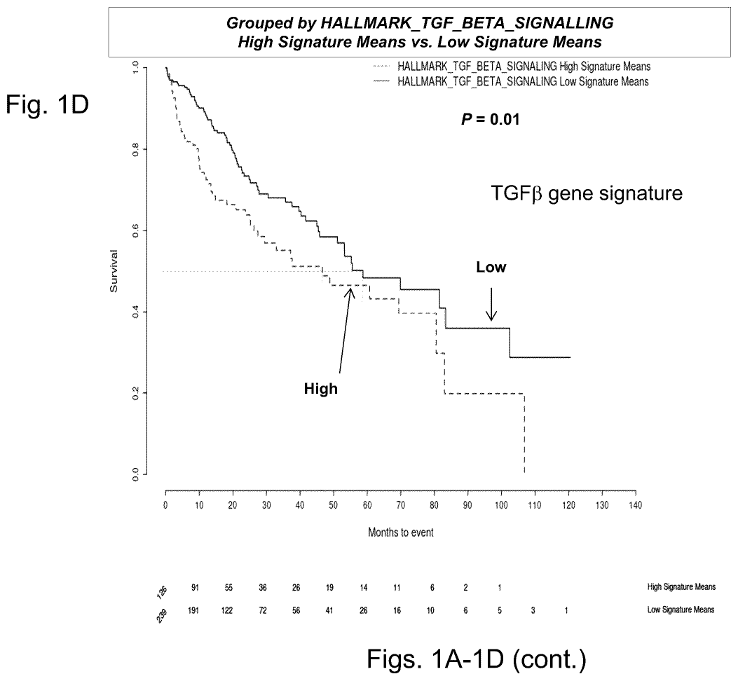

TGFβ1 gene expression was upregulated in primary solid tumor cells (LIHC) 1.74 fold compared to normal tissue. High expression of TGFβ1 in LIHC was associated with decreased OS compared to LIHC expressing low levels of TGFβ1 (median, 47 months and 70 months, respectively). See FIGS. 1A and 1B.

TGFβ gene signaling was increased in primary solid tumors 1.1317 fold compared to normal tissue. High TGFβ signaling in LIHC was associated with decreased cell survival compared to low TGFβ signaling. See FIGS. 1C and 1D.

### Conclusion

These results demonstrate a statistically significant correlation between increased TGFβ1 gene expression and TGFβ signaling with decreased overall survival in patients with LIHC. Therefore, greater TGFβ1 gene expression and TGFβ signaling may play a causative role in cancer related mortality.

### Example 2: TGF-p and Signaling in Liver Hepatocellular Carcinoma

### Summary

In the present example, the expression of TGFβ and the intensity of TGFβ signaling (p-SMAD2) were compared in normal liver versus liver hepatocellular carcinoma (HCC) by immunohistochemistry analysis.

### Methods

Three normal liver samples and 32 Hepatocellular carcinoma samples were stained by immunhistochemistry and the intensity scored. TGF-β1 and pSMAD2 immunohistochemistry were performed on the Ventana Discovery platform using anti-TGF-β1 (Abcam) and anti-pSAMD2 (Cell Signaling Technology) antibodies. TGF-P 1 and pSMAD2 expression in FFPE normal liver and HCC specimens was scored semi-quantitatively by a pathologist. IHC staining intensity was define as: score 0, negative staining; score 1, minimal staining; score 2, moderate staining; score 3, strong staining.

### Results

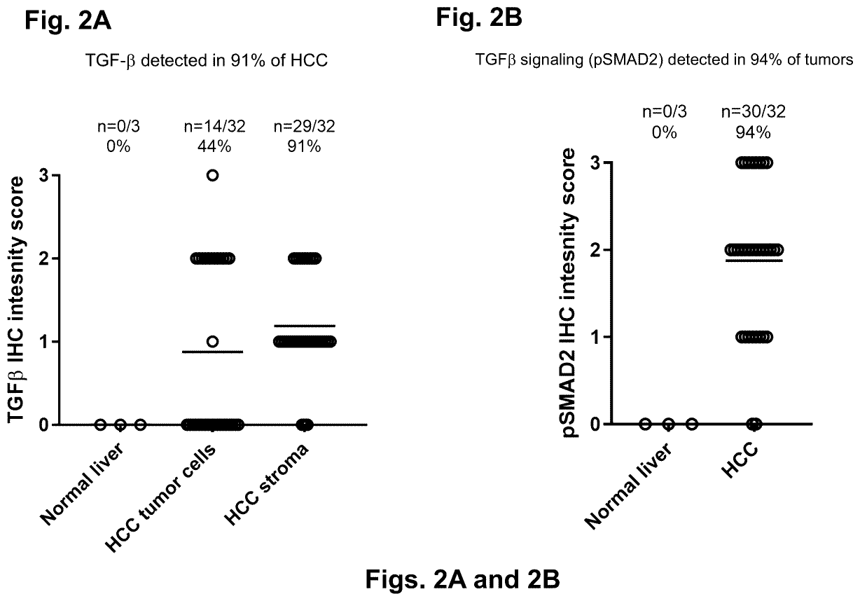

TGF-β was detected in 44% of tumor and 91% of stroma of HCC samples. TGF-β signaling, judged by the intensity phospo-SMAD2 (p-SMAD2), was tected in 91% of HCC samples. Normal liver was negative for both TGF-β and pSMAD-2 (FIGS. 2A and B).

### Conclusions

TGF-β was expressed and actively signaling in the vast majority of HCC samples, demonstrating that it is common immune suppressive factor in HCC tumors and that armoring against it can benefit a large population of HCC patients.

### Example 3: Armoring of GPC3 CAR-T Cells with TGFβRIIDN

### SUMMARY

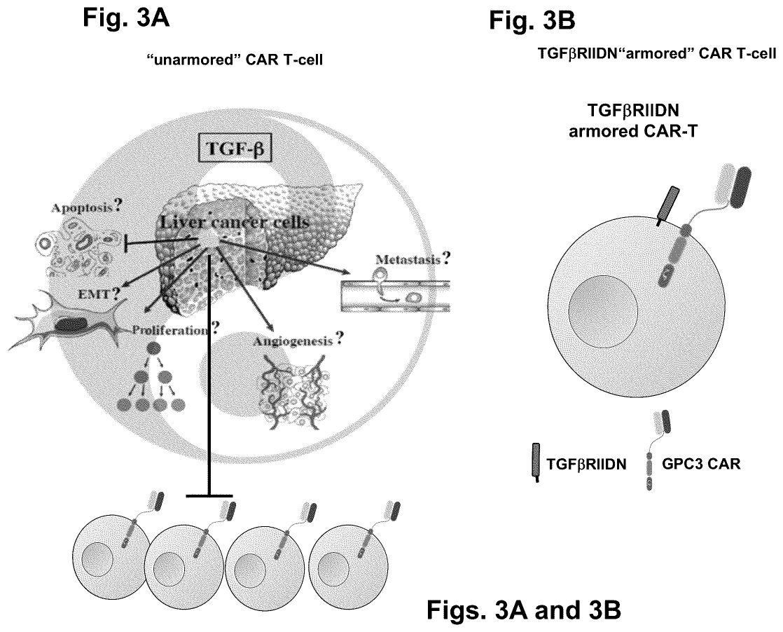

In the present example, armoring of GPC3 BZ CAR-T cells with TGFβRIIDN was examined as a potential way to protect CAR-T cells against TGFβ-mediated immunosuppression to improve CAR-T cell effector function and tumor control. See FIGS. 3A and 3B.

### Methods

TGFβRIIDN: Dominant-negative TGF-β receptor type 2 molecules were prepared by truncating the wild-type receptor at residue 194 so that the TGFβRIIDN receptor lacked an intracellular signaling domain.

Armored CAR-T cells: GPC3 BZ CAR-T cells were armored with TGFβRIIDN by expressing the TGFβRIIDN receptor as a C-terminal fusion to GPC3 BZ CAR with a T2A peptide separating the GPC3 BZ CAR and the TGFβRIIDN receptor.

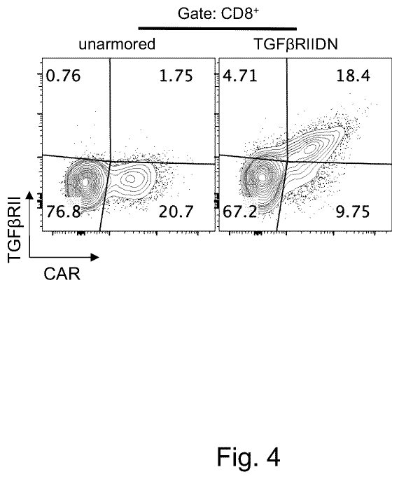

Upon expansion, the expression of CAR and TGFβRII were analyzed on the surface of unarmored and armored CAR-T cells by flow cytometry. CAR expression was detected by using an AF647 anti-idiotypic antibody to the GPC3-CAR (FIG. 4).

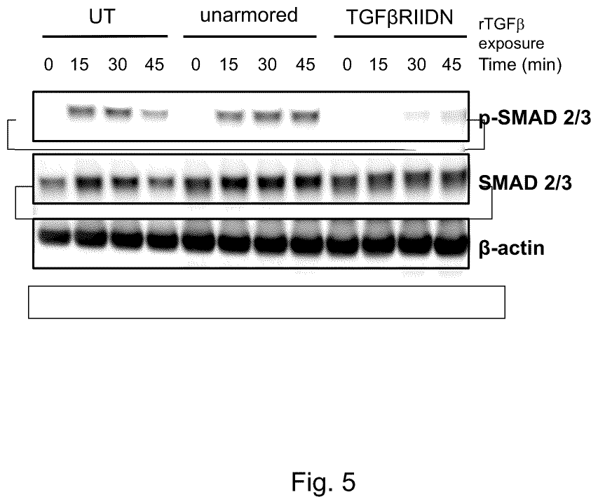

CAR-T cells were purified with anti-AF647 microbeads (Miltenyi) after staining with AF647 anti-idiotypic antibody to the GPC3-CAR. Purified cells were expanded for 5 more days, rested for overnight in the absence of IL-2 and serum, and stimulated with recombinant human TGF-β (1 ng/mL) for the indicated time. Cells were lysed in RIPA buffer containing proteases and phosphatases inhibitors and the expression of the indicated proteins analyzed by western blot. (FIG. 5).

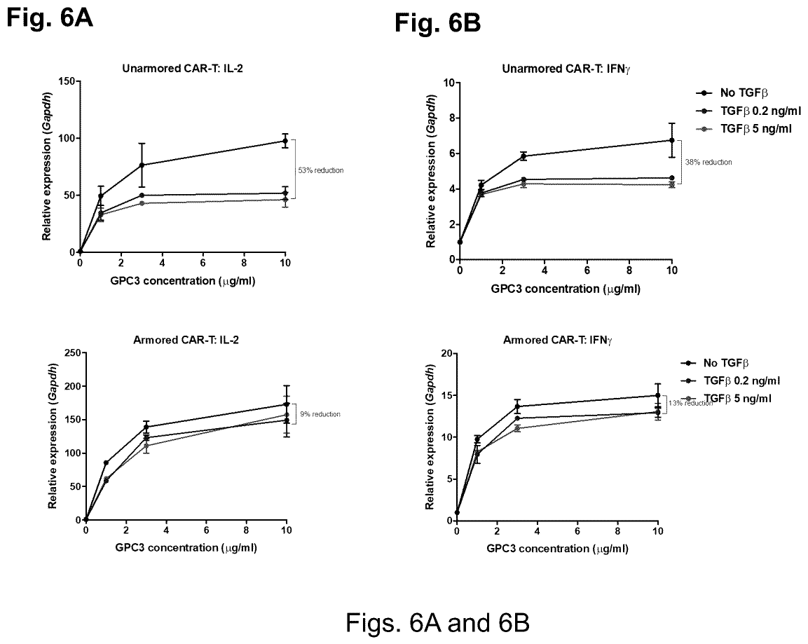

Unarmored or armored CAR-T cells were purified with anti-AF647 microbeads (Miltenyi) after staining with AF647 anti-idiotypic antibody to the GPC3-CAR. Purified cells were stimulated with plate-bound recombinant human GPC3 at the indicated concentration in the presence or absence of recombinant human TGF-β (0.2 or 5 ng/mL). After 6 hours cell were harvested, and total RNA was derived from cells using RNeasy Mini Kit (QIAGEN) and reversed transcribed using High Capacity cDNA Reverse Transcription Kit (Applied Biosystems). Quantitative real-time PCR was performed according to the protocol of TaqMan Gene Expression Master Mix (Applied Biosystems) with the following TaqMan primers: GAPDH, Hs02758991_m1; IL2, Hs00174114_m1 and IFNG, Hs00989291_m1. (FIG. 6)

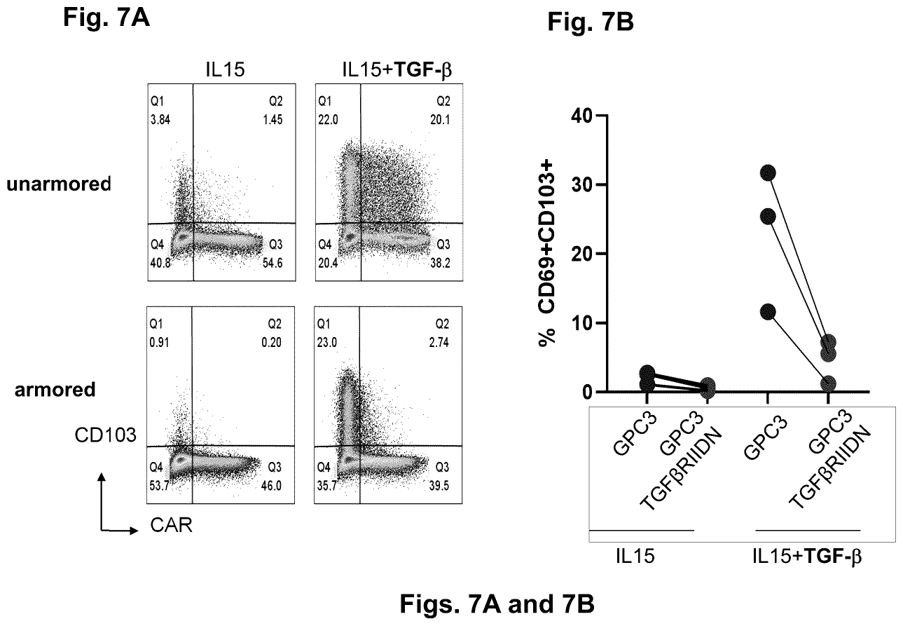

CAR-T cells were stimulated with anti-CD3/CD28 beads (Dynabeads) in the presence of 20 ng/mL of IL-15, without IL-2. After 3 days, TGFβ (50 ng/mL) was added and the cells were assessed for the expression of CD103 by flow cytometry after 3 more days of culture (FIG. 7).

### Results

Expression of TGFβRII on the surface of TGFβRIIDN CAR-T. Surface staining of TGFβRII on unarmored and armored CAR-T was evaluated by flow cytometry. The antibody does not distinguish between endogenous and DN TGFβRII since the extracellular portion is the same, but TGFβRIIDN CAR-T co-expressed TGFβRII and CAR, suggesting that the antibody is detecting the overexpressed DN receptor. See FIG. 4.

TGFβRIIDN inhibits SMAD 2/3 phosphorylation in CAR-T cells upon exposure to rhTGFβ: unarmored CAR-T cells demonstrated rhTGF-β-induced SMAD 2/3 phosphorylation similar to untransduced control cells at 0, 15, 30, and 45 minutes post-rhTGF-β exposure. TGF-βRIIDN armored CAR-T cells demonstrated attenuated SMAD 2/3 phosphorylation compared to unarmored CAR-T cells and untransduced control cells at 15, 30, and 45 minutes following rhTGF-β exposure. Total SMAD 2/3 protein and 3-actin expression was consistent among all groups. See FIG. 5B.

Expression of TGFβRIIDN prevents TGF-β-mediated decrease of effector cytokine production. Stimulation with recombinant human GPC-3 induced the transcription of the effector cytokines IFN-γ and IL-2 in CAR-T cells. When present during the stimulation, TGF-β decreased the level of IFN-γ and IL-2 produced in unarmored but not in armored CAR-T cells. This result demonstrates that the expression of TGFβRIIDN protects CAR-T cells from the immunosuppressive effect of TGF-β. See FIGS. 6A and 6B.

Expression of TGFβRIIDN efficiently inhibits TGFβ signaling. Differentiation of CD103⁻ T cells to CD103⁺ T_{RM} requires IL-15 and TGF-β in vitro. Accordingly, TGFβRIIDN CAR-T cells were not able to differentiate into T_{RM} cells after incubation with TGF-β, as compared to CAR-cells or unarmored CAR-T cells. This result demonstrates that the expression of the TGFβRIIDN inhibits TGF-β-induced signaling during extended exposure. See FIGS. 7A and 7B.

### Conclusion

TGFβRIIDN suppresses TGF-β signaling in armored CAR-T cells, prevents TGF-β mediated decrease of effector cytokine production and mutes TGF-β-mediated differentiation of TGFβRIIDN armored CAR-T cells to a T_{RM} phenotype. Overall these results demonstrate that expression of the dominant negative TGFβRII is sufficient to inhibit TGF-β signaling and its biological effects.

### Example 3: TGFβRIIDN Armored CAR-T Cell Cytotoxicity and Expansion During Co-Culture with GPC3⁺ Cells

### Summary

In the present example, CAR-T cell-mediated cytotoxicity and expansion were compared among UT, unarmored, and TGFβRIIDN-armored CAR-T cells during co-culture with GPC3+ liver cancer cells.

### Methods

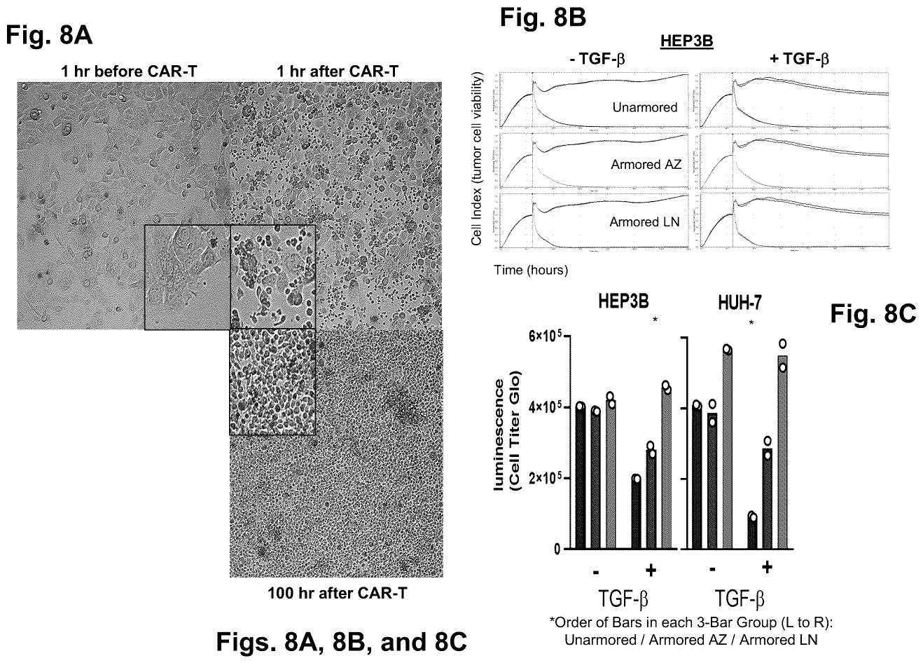

T cells (20,000 CAR+ cells/well) were co-cultured with a squamous cell carcinoma line engineered to express GPC3 (OE21 cells, 10,000 tumor cells per well) for 5 days on an xCELLigence eSight RTCA to simultaneously monitor real-time tumor cell viability by electrical impedance and CAR-T cell density by microscopy (See FIG. 8A). T cells (60,000 CAR+ cells/well) were then co-cultured with Hep3B cells (medium/low GPC3 expression) or Huh7 cells (low GPC3 expression) cells (30,000 tumor cells/well) for 5 days on an xCELLigence RTCA-MP (without microscopy) for 5 days (See FIG. 8B). Non-adherent cells were then removed from the wells and viable CAR-T were quantified using the CellTiter-Glo® Luminescent Cell Viability Assay (Promega, Madison, WI) (See FIG. 8C).

### Results

Dramatic expansion of CAR-T cells was observed while in co-culture with GPC3+ tumor cells. See FIG. 8A. All CAR-T cells efficiently killed GPC3+ tumor cells and cytolytic ability was not modulated by TGFβRIIDN co-expression or by addition of exogenous TGF-β. See FIG. 8B. TGF-β suppressed tumor GPC3-induced proliferation of CAR-T cells after 5 days in co-culture. However, TGFβRIIDN armored CAR-T cells were not susceptible to this suppression. See FIG. 8C.

### Conclusion

TGFβRIIDN-armored CAR-T cells exhibited significant GPC3+ tumor cell cytotoxicity, indicating that expression of the dominant negative receptor did not affect the ability of CAR-T to kill target cells in vitro. Conversely, unlike unarmored CAR-T cells, TGFβRIIDN armored CAR-T cells were not susceptible to TGF-β-mediated suppression of GPC3⁺-induced proliferation.

### Example 4: TGFβRIIDN Armored CAR-T-Cells In Vivo—Xenograft Model

### Summary

In the present example, the effectiveness of TGFβRIIDN armored T cells against GPC3⁺ tumor cells was determined in vivo.

### Methods

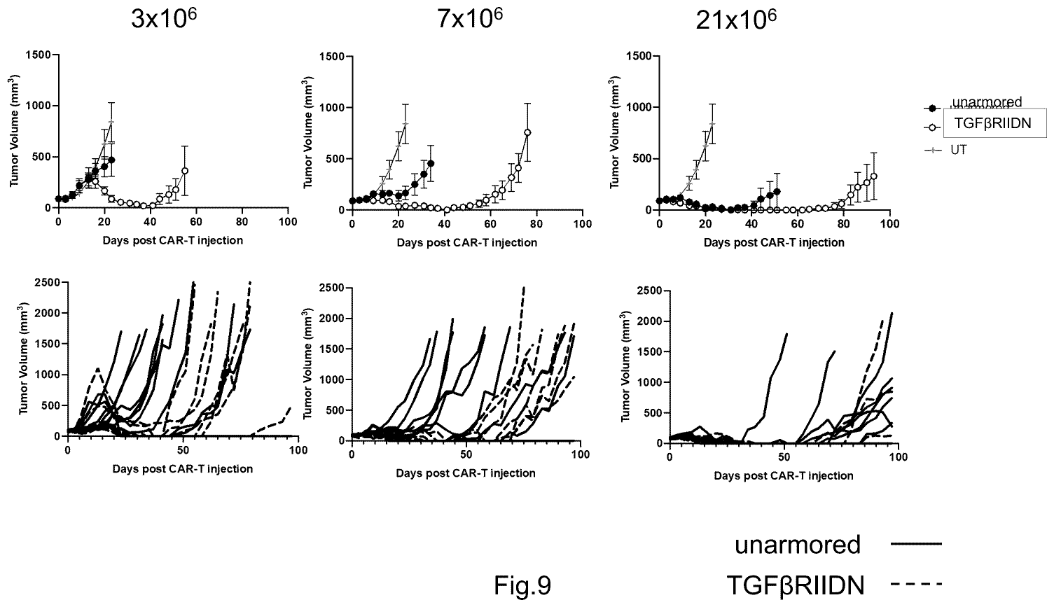

The hepatocellular carcinoma Huh7-TGF-β model, which overexpresses TGF-β was used to test the in vivo effectiveness of TGFβRIIDN-armored T cells in reducing tumor volume. Tumor cells were implanted in the flank of NSG mice (10 mice/group). When tumors reached an average volume of 150 mm³, mice were dosed with 3, 7, or 21×10⁶ of the indicated CAR-T or 21 million of untransduced T cells and tumors measured bi-weekly. Upper graphs: average tumor volume per group. Lower graphs: tumor volume for each individual mouse at the indicated dose (See FIG. 9).

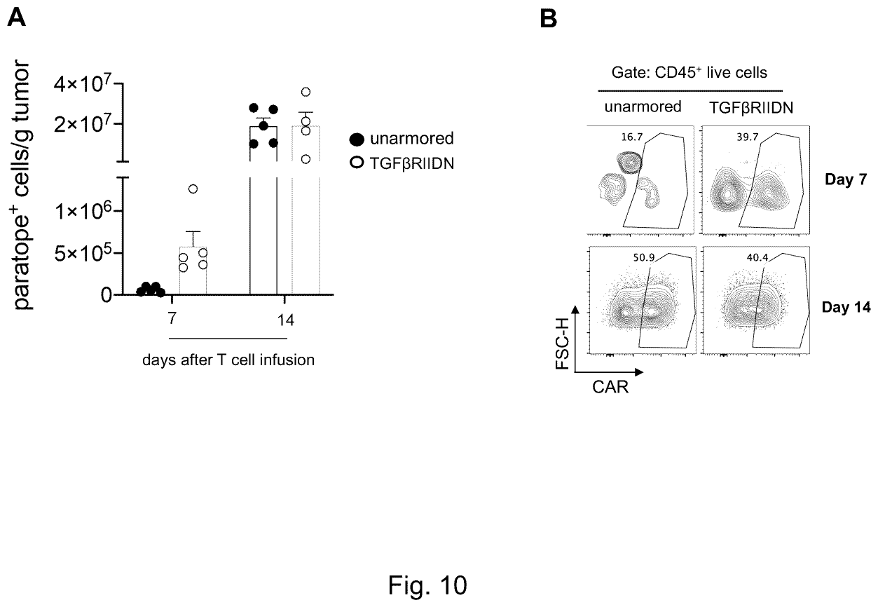

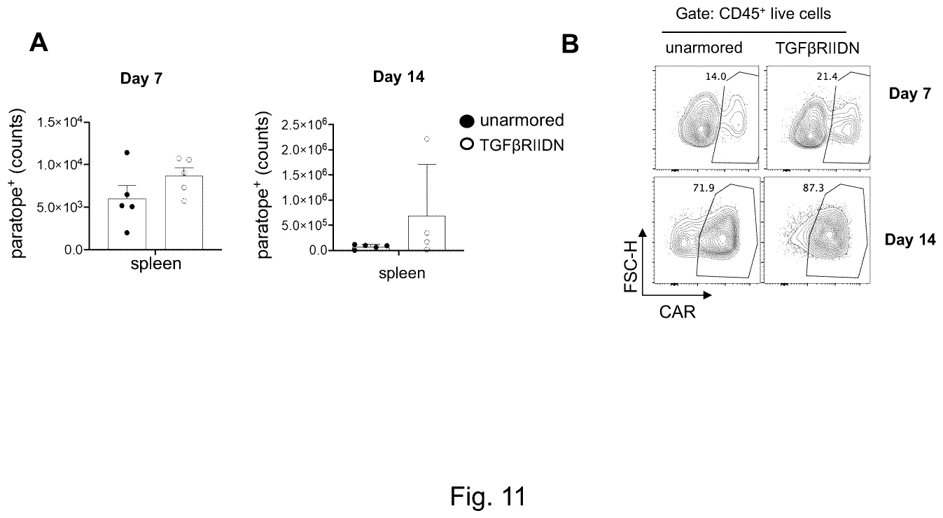

Ex vivo analysis was performed on Huh7-TGFβ tumor bearing mice dosed with 7×10⁶CAR-T. Seven or fourteen days after infusion, tumors and spleens were collected from 5 mice per group. The number of CAR+cells was calculated by flow cytometry upon staining with AF647-labeled anti-idiotypic antibody to the GPC3-CAR and using AccuCheck Counting Beads. (See FIGS. 10 and 11).

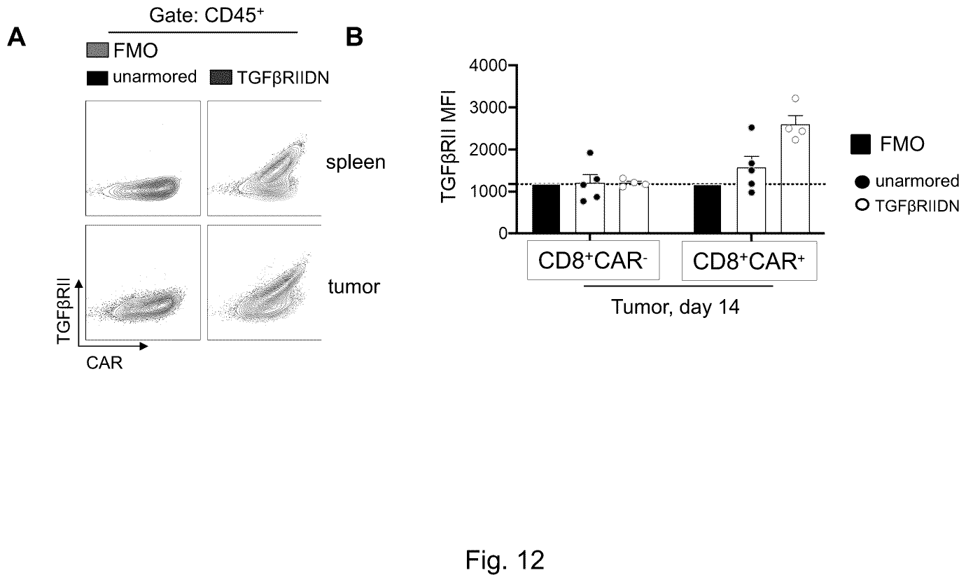

Mice bearing Huh-7-TGFβ tumors were dosed with 7×10⁶CAR-T cells. Fourteen days after infusion, tumors were collected and the expression of the TGFβRII was evaluated by flow cytometry staining on CAR positive and CAR negative cells. (See FIG. 12).

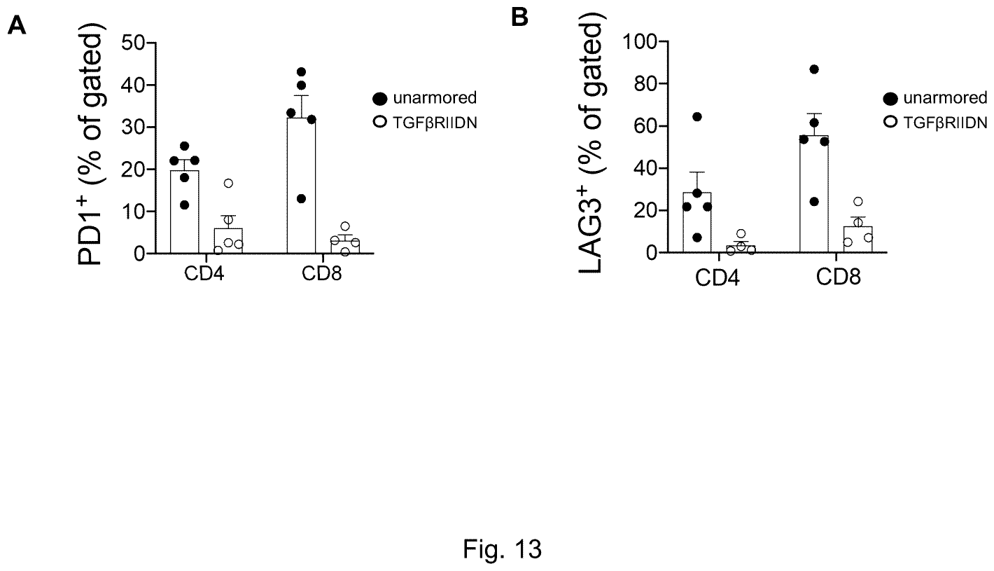

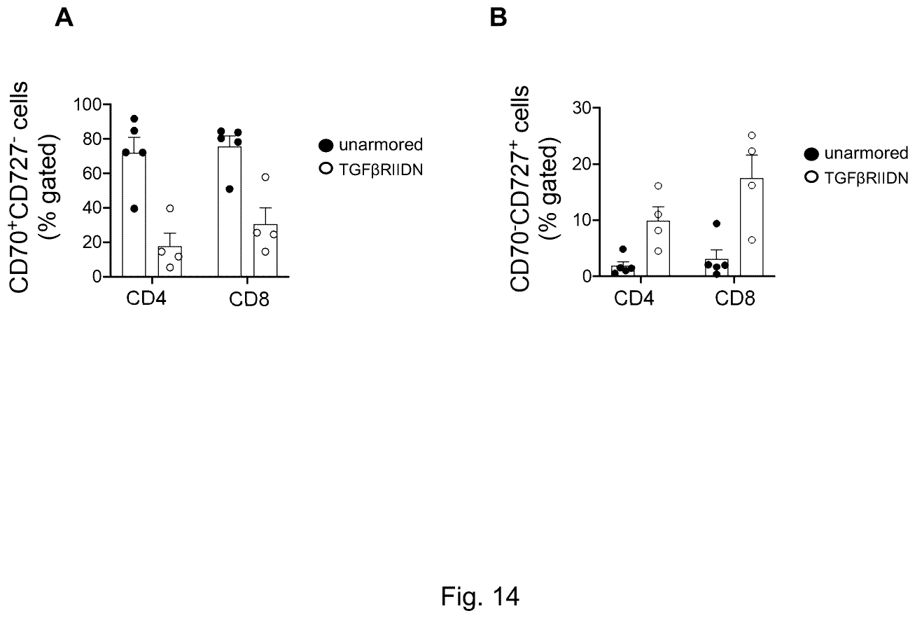

Mice bearing Huh-7-TGFβ tumors were dosed with 7×10⁶CAR-T cells. Fourteen days after infusion, tumors were collected and the expression of PD1, LAG3, CD27 and CD70 was evaluated on the surface of CAR-T. (See FIGS. 13 and 14).

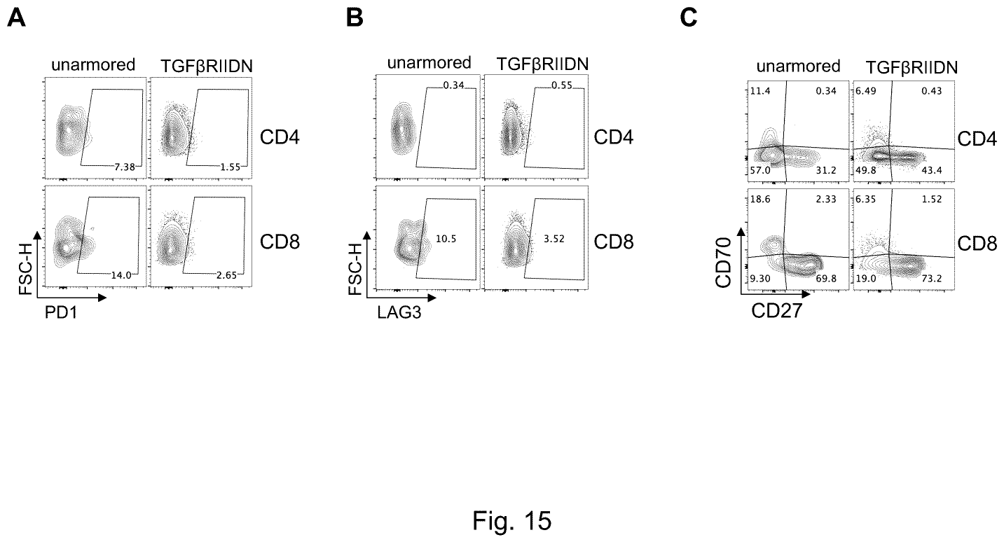

Mice bearing Huh-7-TGFβ tumors were dosed with 7×10⁶CAR-T cells. Fourteen days after infusion, spleens were collected and the expression of PD1, LAG3, CD27 and CD70 was evaluated by flow cytometry staining. (See FIG. 15).

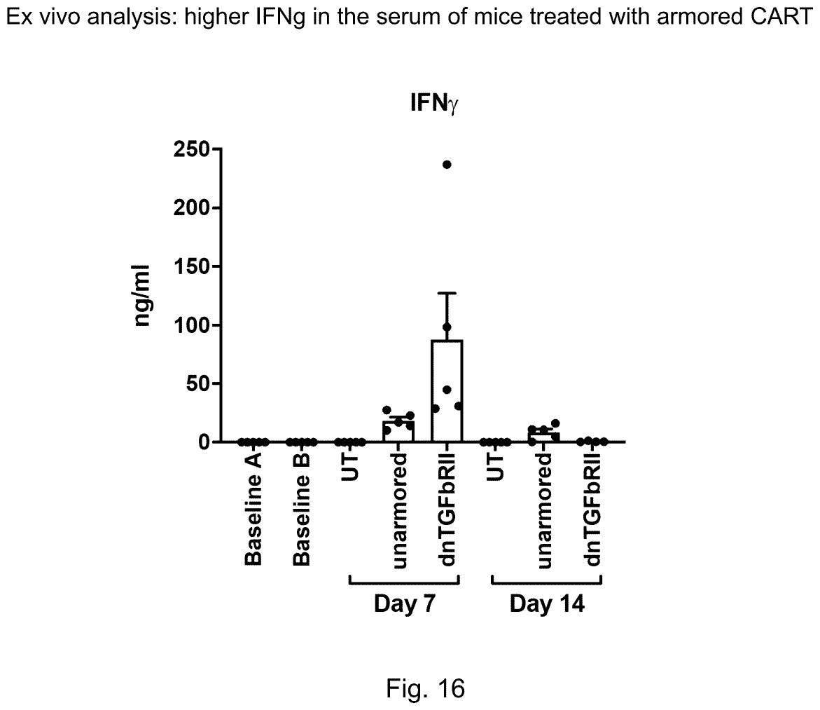

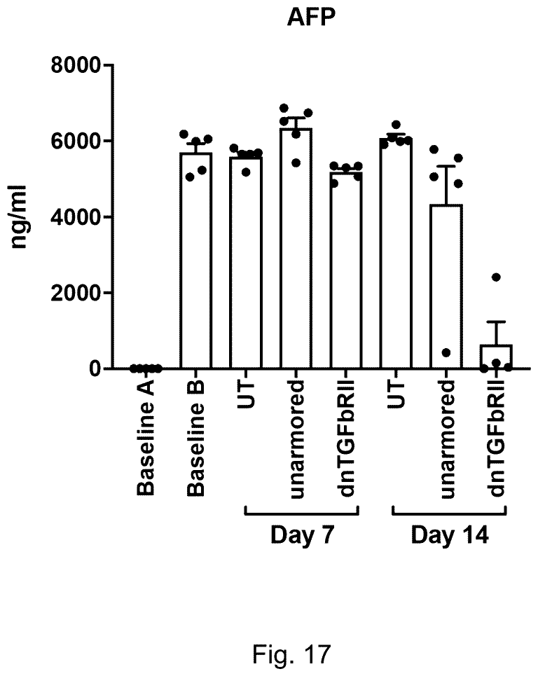

Mice bearing Huh-7-TGFβ tumors were dosed with 7×10⁶CAR-T cells. For serum cytokine and AFP analysis, blood was harvested in small volumes at the indicated time points and serum separated using BD Microtainer Serum Separator Tubes. Cytokine levels were determined using MSD assays, while AFP was assessed by sandwich ELISA. Five mice per group were bled before tumor implantation (Baseline A), before CAR-T infusion (Baseline B), 7- and 14-days post infusion. (See FIGS. 16 and 17).

### Results

Untransduced T cells had no discernable effect on tumor growth. Treatment with unarmored CAR-T cells resulted in minor reduction in tumor volume at the lowest dose (3 million cells/mouse) while a more profound effect was evident at higher doses, resulting in complete tumor regression. In contrast, administration of TGFβRIIDN armored CAR-T cells induced a profound reduction of tumor volume and complete regression even at the lowest dose. Moreover, progression-free survival was significantly extended at all treatment doses of TGFβRIIDN armored CAR-T cell compared to unarmored CAR-T (See FIG. 9). Ex vivo analysis of CAR-T cells was performed on mice infused with 7×10⁶ cells. In line with the enhanced efficacy, an increased number of tumor-infiltrating lymphocytes (TILs) was detected in the TGFβRIIDN treated mice 7 days after infusion, indicating an ongoing active immune response and associated proliferation that plateaued at day 14 post-treatment (See FIG. 10). Conversely, increased number of CAR-T cells was observed 14 days after infusion in the spleen of TGFβRIIDN armored CAR-T-treated mice, suggesting enhanced proliferation by the CAR-T cells and more CAR⁺ cells were therefore detectable in the circulation (See FIG. 11). To investigate if the TGFβRIIDN was detectable on CAR-T cells upon in vivo expansion, we analyzed the expression of TGFβRII on TILs and lymphocytes in the spleen 14 days after infusion. While TGFβRII was barely detectable on unarmored cells, lymphocytes of mice treated with the armored CAR-T cells co-expressed CAR and TGFβRII. Therefore, it is reasonable to assume that the TGFβRII expressed on the TGFβRIIDN TILs is the dominant negative receptor, detectable ex vivo after an active immune response and associated antigen-dependent proliferation (See FIG. 12). Notably, TGFβRIIDN TILs expressed low level of the exhaustion markers LAG3 and PD1 (See FIG. 13), the latter being directly regulated by TGF-β in a SMAD3-dependent fashion (Park, B. V., Freeman, Z. T., Ghasemzadeh, A., Chattergoon, M. A., Rutebemberwa, A., Steigner, J. et al. (2016). TGFβ1-Mediated SMAD3 Enhances PD-1 Expression on Antigen-Specific T Cells in Cancer. Cancer Discov, 6(12), 1366-1381). Moreover, TGFβRIIDN TILs expressed less CD70 and conversely more CD27 compared to unarmored TILs (See FIG. 14). This result agrees with previous evidence showing that TGF-β upregulates CD70 expression and induces exhaustion of effector memory T cells, and reinforce the comcept that expression of the dominant negative receptor protects CAR-T cells from TGF-R mediated immunosuppression (Yang, Z Z., Grote, D., Xiu, B. et al. TGF-β upregulates CD70 expression and induces exhaustion of effector memory T cells in B-cell non-Hodgkin's lymphoma. Leukemia 28, 1872-1884 (2014). https://doi.org/10.1038/leu.2014.84). In contrast to TILs, CAR-T cells in the periphery did not express co-inhibitory markers or CD70 and were mostly CD27 positive consistently with a non-activated status (See FIG. 15). In line with enhanced efficacy and higher number of TILs, more IFN-γ was detected in the serum of mice infused with TGFβRIIDN CAR-T 7 days after infusion, and a dramatic decrease in the serum concentration of the tumor marker AFP 14 days after infusion (See FIGS. 16 and 17). This result demonstrates that expression of TGFβRIIDN increases the efficacy of CAR-T therapy by counteracting the immunosuppressive action of TGF-β in vivo.

### Conclusion

TGFβRIIDN armored CAR-T cells exhibit considerable promise for being an effective treatment for GPC3+ tumors in vivo.

### Example 5: TGFβRIIDN Armored CAR-T-Cells In Vivo—Hepatocellular Carcinoma Patient-Derived Xenograft Models

### Summary

In the present example, the effectiveness of TGFβRIIDN armored CAR-T cells in a number of GPC3⁺ hepatocellular carcinoma patient derived xenograft cells was determined in vivo.

### Methods

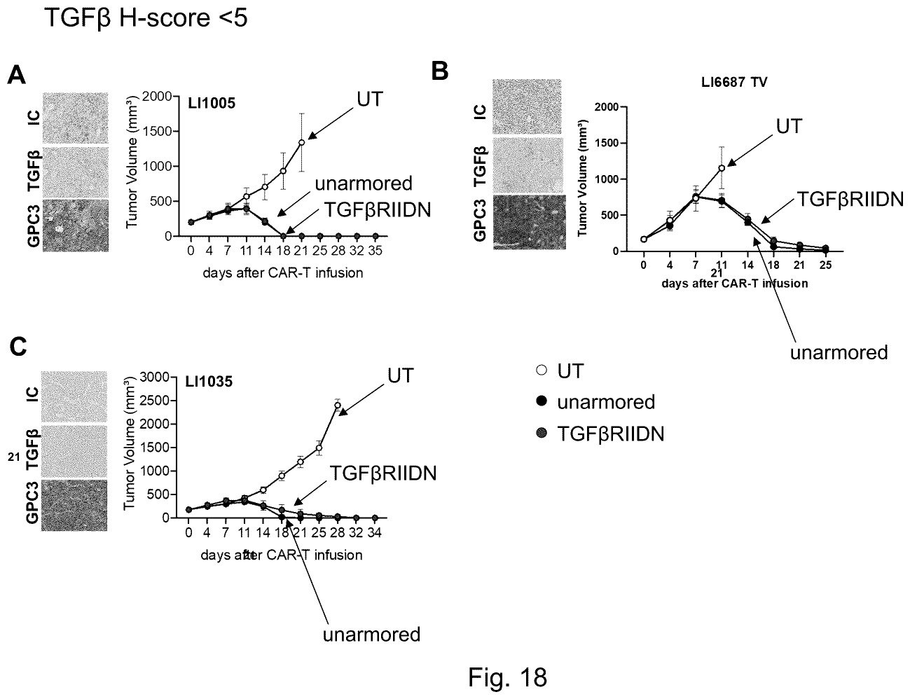

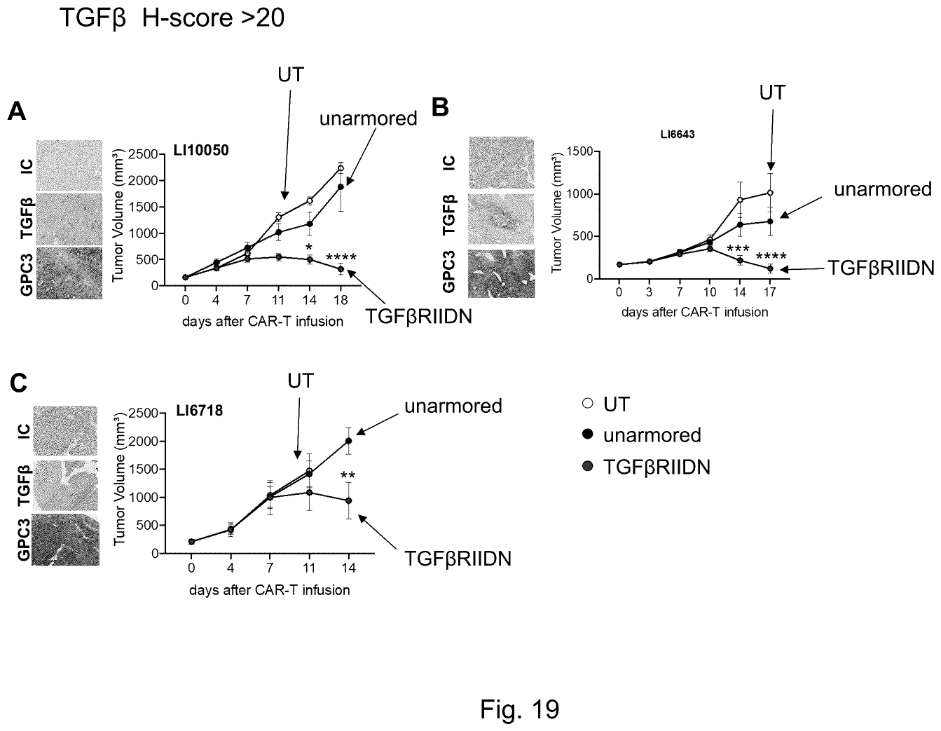

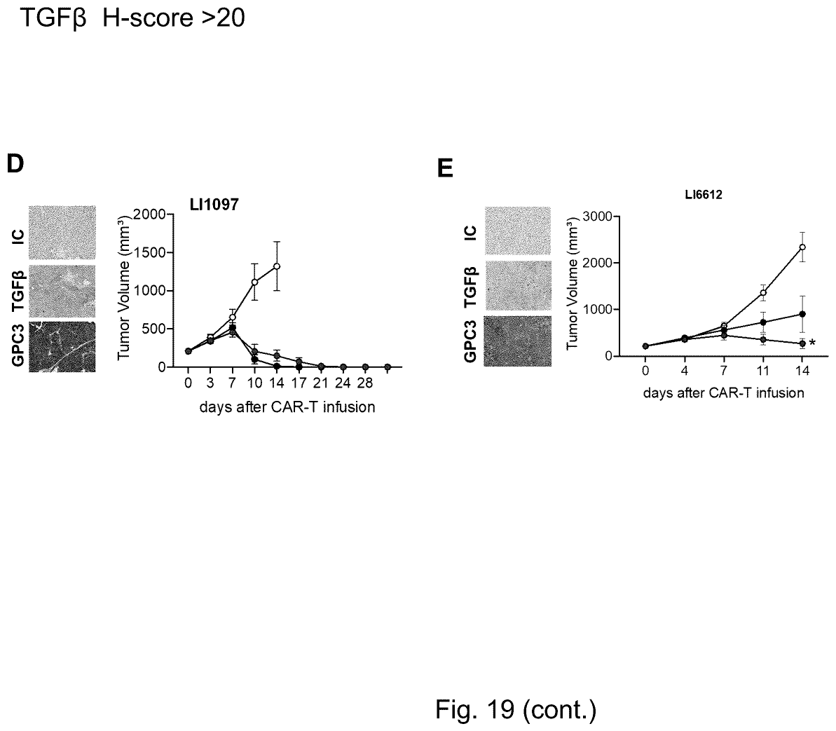

This study was conducted by Crown Bioscience Inc. Eight PDX models were selected based on expression of GPC3 and TGF-β evaluated by IHC and RNA-sequencing, performed by Crown Bioscience. All the models selected were GPC3 high (IHC score >100), but three of them expressed no TGF-β (IHC score <5) while the other 5 models were TGF-β positive (IHC score >20). According to Crown Bioscience study protocol, tumor fragments from stock mice were harvested and used for inoculation into NCG mice. Each mouse was inoculated subcutaneously in the right front flank with a specific PDX tumor fragment (3×3×3 mm) for tumor development. Mice were randomized when the mean tumor size reached approximately 150-250 mm³. Tumor bearing mice were dosed with 5 million of unarmored or armored CAR-T cells, and untransduced T cells provided by AstraZeneca (5 mice/group), and tumor volume was measured bi-weekly. (See FIGS. 18 and 19).

### Results

Tumors grew in the mice that received untransduced T cells. However, in the absence of TGF-β, unarmored and armored cells were equally effective and induced fast and complete regression of all the mice included in the study (See FIG. 18). When infused in TGF-β expressing models, unarmored CAR-T cells had a much less pronounced effect, in contrast TGFβRIIDN armored CAR-T cells were consistently more potent and induced significant tumor regression.

### Conclusion

PDX models simulate human tumor biology allowing for natural cancer progression. Therefore, these observations confirm and reinforce the evidence obtained with the Huh7-TGF-β xenograft model. Overall, these data demonstrate that TGFβRIIDN armored GPC3 CAR-T cells might be an effective treatment for GPC3⁺ tumors in vivo and can maintain their efficacy even in the presence of the immune suppressive factor TGF-β.

The embodiments described herein can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present invention.

TABLE 5
Sequences used in the Examples.

| | |
|---|---|
| SEQ ID NO: 1 | GPC3 scFv amino acid sequence |
| SEQ ID NO: 2 | GPC3-2 scFv amino acid sequence |
| SEQ ID NO: 3 | GPC3 BZ CAR amino acid sequence |
| SEQ ID NO: 4 | GPC3 TZ CAR amino acid sequence |
| SEQ ID NO: 5 | GPC3 28Z CAR amino acid sequence |
| SEQ ID NO: 6 | GPC3 28 BZ CAR amino acid sequence |
| SEQ ID NO: 7 | GPC3-2 BZ CAR amino acid sequence |
| SEQ ID NO: 8 | GPC3-2 TZ CAR amino acid sequence |
| SEQ ID NO: 9 | GPC3-2 28Z CAR amino acid sequence |
| SEQ ID NO: 10 | GPC3-2 28BZ CAR amino acid sequence |
| SEQ ID NO: 11 | GPC3 BZ CAR nucleic acid sequence |
| SEQ ID NO: 12 | GPC3 TZ CAR nucleic acid sequence |
| SEQ ID NO: 13 | GPC3 28Z CAR nucleic acid sequence |
| SEQ ID NO: 14 | GPC3 28BZ CAR nucleic acid sequence |
| SEQ ID NO: 15 | GPC3-2 BZ CAR nucleic acid sequence |
| SEQ ID NO: 16 | GPC3-2 TZ CAR nucleic acid sequence |
| SEQ ID NO: 17 | GPC3-2 28Z CAR nucleic acid sequence |
| SEQ ID NO: 18 | GPC3-2 28BZ CAR nucleic acid sequence |
| SEQ ID NO: 19 | GPC3-3 BZ CAR amino acid sequence |
| SEQ ID NO: 20 | GPC3-3 28BZ CAR amino acid sequence |
| SEQ ID NO: 21 | GPC3-4 BZ CAR amino acid sequence |
| SEQ ID NO: 22 | GPC3-3 BZ CAR nucleic acid sequence |
| SEQ ID NO: 23 | GPC3-3 28BZ CAR nucleic acid sequence |
| SEQ ID NO: 24 | GPC3-4 CAR nucleic acid sequence |
| SEQ ID NO: 25 | GPC3 BZ CAR amino acid |
| | sequence (WPRE-deleted) |
| SEQ ID NO: 26 | GPC3 BZ CAR nucleic acid |
| | sequence (WPRE-deleted) |
| SEQ ID NO: 27 | GPC3 VH |
| SEQ ID NO: 28 | GPC3 VL |
| SEQ ID NO: 29 | GPC3-2 VH |
| SEQ ID NO: 30 | GPC3-2 VL |
| SEQ ID NO: 31 | GPC3-3 scFv amino acid sequence |
| SEQ ID NO: 32 | GPC3-4 scFv amino acid sequence |
| SEQ ID NO: 33 | GPC3 scFv nucleic acid sequence |
| SEQ ID NO: 34 | GPC3-2 scFv nucleic acid sequence |
| SEQ ID NO: 35 | GPC3-3 scFv nucleic acid sequence |
| SEQ ID NO: 36 | GPC3-4 scFv nucleic acid sequence |
| SEQ ID NO: 37 | GPC3 and GPC3-2 VH CDR1 |
| SEQ ID NO: 38 | GPC3 and GPC3-2 VH CDR2 |
| SEQ ID NO: 39 | GPC3 and GPC3-2 VH CDR3 |
| SEQ ID NO: 40 | GPC3 VL CDR1 |
| SEQ ID NO: 41 | GPC3 VL CDR2 |
| SEQ ID NO: 42 | GPC3 VL CDR3 |
| SEQ ID NO: 43 | GPC3-2 VL CDR1 |
| SEQ ID NO: 44 | GPC3-2 VL CDR2 |
| SEQ ID NO: 45 | GPC3-2 VL CDR3 |
| SEQ ID NO: 46 | GPC3 BZ CAR and Dominant-negative |
| | TGF-β receptor type 2 nucleic acid sequence |
| SEQ ID NO: 47 | GPC3 BZ CAR and Dominant-negative |
| | TGF-β receptor type 2 amino acid sequence |

TABLE 6
Sequences

| | |
|---|---|
| SEQ ID NO: 1 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCARGKRYFDYWGQGTMVTVSSGGGGSGGG |
| | GSGGGGSSYELTQPPSASGTPGQRVTISCSGGSSNIGSNTVNW |
| | FRQLPGTAPKLLVYFNNQRPSGVPDRFSGSKSGTSASLAIGGL |
| | QSDDEADYYCVAWDDSLNAPVFGGGTKVTVL |
| SEQ ID NO: 2 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCAKGKRYFDYWGQGTMVTVSSGGGGSGGG |
| | GSGGGGSQSVLTQPPSASGTPGQRVTISCSGGSSDIGSNTVNW |
| | YQQLPGTAPKLLIYYNNQRPSGVPDRFSGSKSGTSASLAISGL |
| | QSEDEADYYCATWDDRMYSPVFGGGTKLTVL |
| SEQ ID NO: 3 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVKRGRKKLLYIFKQPFMRPVQTTQEEDGCSCR |
| | FPEEEEGGCELRVKFSRSADAPAYQQGQNQLYNELNLGRREE |
| | YDVLDKRRGRDPEMGGKPRRKNPQEGLYNELQKDKMAEAY |
| | SEIGMKGERRRGKGHDGLYQGLSTATKDTYDALHMQALPPR |
| | * |
| SEQ ID NO: 4 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVRVKFSRSADAPA* |
| SEQ ID NO: 5 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVRSKRSRLLHSDYMNMTPRRPGPTRKHYQPY |
| | APPRDFAAYRSRVKFSRSADAPAYQQGQNQLYNELNLGRRE |
| | EYDVLDKRRGRDPEMGGKPRRKNPQEGLYNELQKDKMAEA |
| | YSEIGMKGERRRGKGHDGLYQGLSTATKDTYDALHMQALPP |
| | R* |
| SEQ ID NO: 6 | MLLLVTSLLLCELPHPAFLLIPGVHSEVQLLESGGGLVQPGGS |
| | LRLSCAASGFTFSSYAMSWVRQAPGKGLEWVSAISGSGGSTY |
| | YADSVKGRFTISRDNSKNTLYLQMNSLRAEDTAVYYCARGK |
| | RYFDYWGQGTMVTVSSGGGGSGGGGSGGGGSSYELTQPPSA |
| | SGTPGQRVTISCSGGSSNIGSNTVNWFRQLPGTAPKLLVYFNN |
| | QRPSGVPDRFSGSKSGTSASLAIGGLQSDDEADYYCVAWDDS |
| | LNAPVFGGGTKVTVLESKYGPPCPPCPFWVLVVVGGVLACYS |
| | LLVTVAFIIFWVRSKRSRLLHSDYMNMTPRRPGPTRKHYQPY |
| | APPRDFAAYRSKRGRKKLLYIFKQPFMRPVQTTQEEDGCSCR |
| | FPEEEEGGCELRVKFSRSADAPAYQQGQNQLYNELNLGRREE |
| | YDVLDKRRGRDPEMGGKPRRKNPQEGLYNELQKDKMAEAY |
| | SEIGMKGERRRGKGHDGLYQGLSTATKDTYDALHMQALPPR |
| | * |
| SEQ ID NO: 7 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR* |
| SEQ ID NO: 8 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVRVKFSRSADAPA* |
| SEQ ID NO: 9 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVRSKRSRLLHSDYMNMTPRRPGPTRKHYQPYAPPR |
| | DFAAYRSRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR* |
| SEQ ID NO: 10 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCAKGKRYFD |
| | YWGQGTMVTVSSGGGGSGGGGSGGGGSQSVLTQPPSASGTP |
| | GQRVTISCSGGSSDIGSNTVNWYQQLPGTAPKLLIYYNNQRPS |
| | GVPDRFSGSKSGTSASLAISGLQSEDEADYYCATWDDRMYSP |
| | VFGGGTKLTVLESKYGPPCPPCPFWVLVVVGGVLACYSLLVT |
| | VAFIIFWVRSKRSRLLHSDYMNMTPRRPGPTRKHYQPYAPPR |
| | DFAAYRSKRGRKKLLYIFKQPFMRPVQTTQEEDGCSCRFPEEE |
| | EGGCELRVKFSRSADAPAYQQGQNQLYNELNLGRREEYDVL |
| | DKRRGRDPEMGGKPRRKNPQEGLYNELQKDKMAEAYSEIG |
| | MKGERRRGKGHDGLYQGLSTATKDTYDALHMQALPPR* |
| SEQ ID NO: 11 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattctctgctggtcacagtggctttcatcatcttctgggtcaa |
| | gcgaggccggaagaaactgctgtacatcttcaaacagccttttatgcgcccagtgcagacaactc |
| | aggaggaagacggctgctcttgtcggttccccgaggaagaggaagggggatgtgagctgcgcg |
| | tgaagttttctcgaagtgccgatgctcctgcatatcagcagggacagaaccagctgtacaacgagc |
| | tgaatctgggccggagagaggaatacgacgtgctggataagaggcgcggcagagacccagaa |
| | atgggcgggaagccacgacggaaaaacccccaggaggggctgtataatgaactgcagaagga |
| | caaaatggccgaggcttacagcgaaatcgggatgaagggagagagaaggcgcggaaaaggc |
| | cacgatggactgtatcagggcctgagcactgccaccaaggacacctacgatgctctgcacatgca |
| | ggcactgccacccaggtga |
| SEQ ID NO: 12 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattctctgctggtcacagtggctttcatcatcttctgggtccg |
| | cgtgaagttttctcgaagtgccgatgctcctgcatga |
| SEQ ID NO: 13 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattccctgctggtcactgtggccttcatcatcttctgggtgcg |
| | gagcaagcggagccggctgctgcactctgactacatgaacatgactccacggagacccggccct |
| | acccggaaacattatcagccctacgccccacccagagattttgccgcttataggtccagggtgaa |
| | gttttctcgcagtgcagatgcccctgcttatcagcagggacagaatcagctgtacaacgagctgaa |
| | tctgggcaggcgcgaggaatacgacgtgctggataagcgacggggcagagaccccgaaatgg |
| | gagggaagcccagaaggaaaaaccctcaggaggggctgtataatgaactgcagaaggacaaa |
| | atggcagaggcctacagtgaaatcgggatgaagggagagcgccgacggggaaaaggccacg |
| | atggactgtatcagggcctgtctactgccaccaaggacacctacgatgccctgcacatgcaggct |
| | ctgcctccacgctga |
| SEQ ID NO: 14 | atgctgctgctggtgacaagcctgctgctgtgcgaactgccccatcccgccttcctgctgattcctg |
| | gtgtacactccgaggtgcagctgttggagtctgggggaggcttggtacagcctggggggtccctg |
| | agactctcctgtgcagcctctggattcacctttagcagctatgccatgagctgggtccgccaggctc |
| | cagggaaggggctggagtgggtctcagctattagtggtagtggtggtagcacatactacgcagac |
| | tccgtgaagggccggttcaccatctccagagacaattccaagaacacgctgtatctgcaaatgaac |
| | agcctgagagccgaggacacggccgtgtattactgtgcgagaggaaagcgatactttgactactg |
| | gggccaggggacaatggtcaccgtctcgagtggtggggggggcagcggtggtggaggctctg |
| | gtggaggagggagctcctatgagctgactcagccaccctcagcgtctgggacccccgggcaga |
| | gggtcaccatctcttgttctggaggcagctccaacatcggaagtaatactgtaaactggttccggca |
| | gctcccaggaacggcccccaaactcctcgtttattttaataatcagcgaccctcaggggtccctga |
| | ccgattctctggctccaagtctggcacctcggcctccctggccatcggtgggctccagtctgacga |
| | tgaggctgactattactgtgtagcatgggatgactctctgaatgctccggtgttcggcggagggac |
| | caaggtcaccgtcctagagagcaaatatggaccaccatgccctccatgtcctttttgggtcctggtg |
| | gtcgtgggaggcgtgctggcatgttattccctgctggtcactgtggccttcatcatcttctgggtgcg |
| | gagcaagcggagccggctgctgcactctgactacatgaacatgactccacggagacccggccct |
| | acccggaaacattatcagccctacgccccacccagagattttgccgcttataggtccaagcgcgg |
| | ccgaaagaaactgctgtacatcttcaaacagcccttcatgagacccgtccagacaactcaggagg |
| | aagacggctgcagctgtaggttccccgaggaagaggaagggggatgtgagctgagggtgaagt |
| | tttctcgcagtgcagatgcccctgcttatcagcagggacagaatcagctgtacaacgagctgaatc |
| | tgggcaggcgcgaggaatacgacgtgctggataagcgacggggcagagaccccgaaatggg |
| | agggaagcccagaaggaaaaaccctcaggaggggctgtataatgaactgcagaaggacaaaat |
| | ggcagaggcctacagtgaaatcgggatgaagggagagcgccgacggggaaaaggccacgat |
| | ggactgtatcagggcctgtctactgccaccaaggacacctacgatgccctgcacatgcaggctct |
| | gcctccacgctga |
| SEQ ID NO: 15 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttattctctgctggtcacagtggctttcatcatcttctgggtcaagcgagg |
| | ccggaagaaactgctgtacatcttcaaacagccttttatgcgcccagtgcagacaactcaggagg |
| | aagacggctgctcttgtcggttccccgaggaagaggaagggggatgtgagctgcgcgtgaagtt |
| | ttctcgaagtgccgatgctcctgcatatcagcagggacagaaccagctgtacaacgagctgaatct |
| | gggccggagagaggaatacgacgtgctggataagaggcgcggcagagacccagaaatgggc |
| | gggaagccacgacggaaaaacccccaggaggggctgtataatgaactgcagaaggacaaaat |
| | ggccgaggcttacagcgaaatcgggatgaagggagagagaaggcgcggaaaaggccacgat |
| | ggactgtatcagggcctgagcactgccaccaaggacacctacgatgctctgcacatgcaggcac |
| | tgccacccaggtga |
| SEQ ID NO: 16 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttactccctgctggtcactgtggccttcatcatcttctgggtgcgggtga |
| | agttttctcgcagtgccgacgctcccgcatga |
| SEQ ID NO: 17 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttattccctgctggtcacagtggccttcatcatcttctgggtgcggagca |
| | agcggagccggctgctgcactctgactacatgaacatgaccccccggagacccggccctacaa |
| | gaaagcattatcagccttacgccccacccagggacttcgcagcttatcgctcccgagtgaaattttc |
| | tcgcagtgcagatgcccccgcttatcagcagggccagaatcagctgtacaacgagctgaatctgg |
| | ggaggcgcgaggaatacgacgtgctggataagcgacggggccgggaccccgaaatgggagg |
| | aaagcctagaaggaaaaacccacaggagggcctgtataatgaactgcagaaggacaaaatggc |
| | agaggcctacagcgaaatcggaatgaagggagagcgccgacggggcaaaggacacgatggc |
| | ctgtatcaggggctgagcaccgccacaaaggacacctacgatgccctgcacatgcaggctctgc |
| | ctccacgctga |
| SEQ ID NO: 18 | atgctgctgctggtgacaagcctgctgctgtgcgaactgccccatcccgccttcctgctgattcctg |
| | aggtccagctgctggagagcggaggaggactggtgcagcctggaggaagtctgcgactgtcat |
| | gcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctggagagcaaatatggaccaccatgccctccatgtcctttttgggtcctggtggtcgtg |
| | ggaggcgtgctggcatgttattccctgctggtcactgtggccttcatcatcttctgggtgcggagca |
| | agcggagccggctgctgcactctgactacatgaacatgactccacggagacccggccctacccg |
| | gaaacattatcagccctacgccccacccagagattttgccgcttataggtccaagcgcggccgaa |
| | agaaactgctgtacatcttcaaacagcccttcatgagacccgtccagacaactcaggaggaagac |
| | ggctgcagctgtaggttccccgaggaagaggaagggggatgtgagctgagggtgaagttttctc |
| | gcagtgcagatgcccctgcttatcagcagggacagaatcagctgtacaacgagctgaatctgggc |
| | aggcgcgaggaatacgacgtgctggataagcgacggggcagagaccccgaaatgggaggga |
| | agcccagaaggaaaaaccctcaggaggggctgtataatgaactgcagaaggacaaaatggcag |
| | aggcctacagtgaaatcgggatgaagggagagcgccgacggggaaaaggccacgatggactg |
| | tatcagggcctgtctactgccaccaaggacacctacgatgccctgcacatgcaggctctgcctcca |
| | cgctga |
| SEQ ID NO: 19 | MLLLVTSLLLCELPHPAFLLIPDVVMTQSPLSLPVTPGEPASIS |
| | CRSSQSLVHSNRNTYLHWYLQKPGQSPQLLIYKVSNRFSGVP |
| | DRFSGSGSGTDFTLKISRVEAEDVGVYYCSQNTHVPPTFGQGT |
| | KLEIKGGGGSGGGGSGGGGSQVQLVQSGAEVKKPGASVKVS |
| | CKASGYTFTDYEMHWVRQAPGQGLEWMGALDPKTGDTAYS |
| | QKFKGRVTLTADKSTSTAYMELSSLTSEDTAVYYCTRFYSYT |
| | YWGQGTLVTVSSDKTHTCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPQRRKNPQEGLYNELQKDKMAEAYSEI |
| | GMKGERRRGKGHDGLYQGLSTATKDTYDALHMQALPPR* |
| SEQ ID NO: 20 | MLLLVTSLLLCELPHPAFLLIPDVVMTQSPLSLPVTPGEPASIS |
| | CRSSQSLVHSNRNTYLHWYLQKPGQSPQLLIYKVSNRFSGVP |
| | DRFSGSGSGTDFTLKISRVEAEDVGVYYCSQNTHVPPTFGQGT |
| | KLEIKGGGGSGGGGSGGGGSQVQLVQSGAEVKKPGASVKVS |
| | CKASGYTFTDYEMHWVRQAPGQGLEWMGALDPKTGDTAYS |
| | QKFKGRVTLTADKSTSTAYMELSSLTSEDTAVYYCTRFYSYT |
| | YWGQGTLVTVSSDKTHTCPPCPFWVLVVVGGVLACYSLLVT |
| | VAFIIFWVRSKRSRLLHSDYMNMTPRRPGPTRKHYQPYAPPR |
| | DFAAYRSKRGRKKLLYIFKQPFMRPVQTTQEEDGCSCRFPEEE |
| | EGGCELRVKFSRSADAPAYQQGQNQLYNELNLGRREEYDVL |
| | DKRRGRDPEMGGKPQRRKNPQEGLYNELQKDKMAEAYSEIG |
| | MKGERRRGKGHDGLYQGLSTATKDTYDALHMQALPPR* |
| SEQ ID NO: 21 | MLLLVTSLLLCELPHPAFLLIPQVQLVQSGGGVVQPGRSLRLS |
| | CAASGFTFSSYGLHWVRQAPGKGLEWVAAISYDGSKKYYAD |
| | SVKGRLTISRDNSKNTLYLQMNSLRPDDTALYFCARGWFVEP |
| | LSWGQGTLVTVSSGGGGSGGGGSGGGGSQSVLTQPPSASGTP |
| | GQRVTISCSGSSSNIGSNTVNWYQQLPGTAPKLLIYSNNQRPS |
| | GVPDRFSGSKSGTSASLAISGLQSEDEADYYCAAWDDSLNGY |
| | VFGTGTKLTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR* |
| SEQ ID NO: 22 | atgctgctgctggtgacaagcctgctgctgtgcgaactgccccatcccgccttcctgctgattcctg |
| | atgtcgtgatgacgcagagccctctctctcttcccgttacccctggtgaacccgcatcaataagttg |
| | ccgctccagtcaatcacttgtacattcaaatcgcaatacctacctgcactggtatttgcagaagccg |
| | ggacaatcccctcaattgttgatatataaggtatccaatcgcttttctggagttcctgatagattcagc |
| | ggatccgggtctggtactgatttcactctgaaaatatccagggtcgaagctgaggacgtaggcgta |
| | tattattgctctcagaacacgcatgtcccgccgactttcggccagggcactaaacttgagatcaagg |
| | gtggggggggcagcggtggtggaggctctggtggaggagggagccaggtccaactcgttcaa |
| | agtggcgcagaggtcaaaaagccaggcgcgagcgttaaagtatcatgtaaggccagcggttata |
| | ctttcactgattatgaaatgcactgggtgcgacaagcccccgggcaaggtcttgagtggatgggtg |
| | cacttgatccaaaaactggggatactgcctatagccagaaattcaaagggcgcgtcacactcactg |
| | ccgacaaaagtacgagcacagcttatatggaattgagttcactgacgagcgaggatacggcagtt |
| | tattactgtacgcgcttctactcttacacttattgggggcaaggcactttggttactgtgtcctctgaca |
| | agacccatacgtgtccaccgtgtcccttctgggtattggttgtggtcggcggtgtccttgcttgttaca |
| | gccttctcgtgacagtcgcattcataattttttgggtgaaaagaggtcggaaaaagttgctgtatatttt |
| | caaacaaccctttatgagacctgtacaaacgactcaggaagaggatggttgtagttgcaggtttcc |
| | ggaggaggaggaaggtgggtgcgaactgcgggtgaaatttagtagaagcgctgacgcaccag |
| | cttaccaacaaggacagaaccaattgtacaacgagcttaacttgggtaggagggaggaatatgat |
| | gtactggacaaaaggcgaggtcgcgatccggaaatgggaggcaagccacagcgccggaaaaa |
| | cccgcaggaaggcttgtacaacgaacttcagaaagataaaatggcagaagcatactccgaaata |
| | gggatgaaaggtgaacggcggcgaggcaagggccacgacggtctgtaccaagggttgtcaac |
| | ggcaactaaagacacgtatgatgcacttcatatgcaagctctgccacccaggtga |
| SEQ ID NO: 23 | atgctgctgctggtgacaagcctgctgctgtgcgaactgccccatcccgccttcctgctgattcctg |
| | atgtcgtgatgacgcagagccctctctctcttcccgttacccctggtgaacccgcatcaataagttg |
| | ccgctccagtcaatcacttgtacattcaaatcgcaatacctacctgcactggtatttgcagaagccg |
| | ggacaatcccctcaattgttgatatataaggtatccaatcgcttttctggagttcctgatagattcagc |
| | ggatccgggtctggtactgatttcactctgaaaatatccagggtcgaagctgaggacgtaggcgta |
| | tattattgctctcagaacacgcatgtcccgccgactttcggccagggcactaaacttgagatcaagg |
| | gtggggggggcagcggtggtggaggctctggtggaggagggagccaggtccaactcgttcaa |
| | agtggcgcagaggtcaaaaagccaggcgcgagcgttaaagtatcatgtaaggccagcggttata |
| | ctttcactgattatgaaatgcactgggtgcgacaagcccccgggcaaggtcttgagtggatgggtg |
| | cacttgatccaaaaactggggatactgcctatagccagaaattcaaagggcgcgtcacactcactg |
| | ccgacaaaagtacgagcacagcttatatggaattgagttcactgacgagcgaggatacggcagtt |
| | tattactgtacgcgcttctactcttacacttattgggggcaaggcactttggttactgtgtcctctgaca |
| | agacccatacgtgtccaccgtgtcccttctgggtattggttgtggtcggcggtgtccttgcttgttaca |
| | gccttctcgtgacagtcgcattcataattttttgggtgcggagcaagcggagccggctgctgcactc |
| | tgactacatgaacatgactccacggagacccggccctacccggaaacattatcagccctacgccc |
| | cacccagagattttgccgcttataggtccaaaagaggtcggaaaaagttgctgtatattttcaaaca |
| | accctttatgagacctgtacaaacgactcaggaagaggatggttgtagttgcaggtttccggagga |
| | ggaggaaggtgggtgcgaactgcgggtgaaatttagtagaagcgctgacgcaccagcttaccaa |
| | caaggacagaaccaattgtacaacgagcttaacttgggtaggagggaggaatatgatgtactgga |
| | caaaaggcgaggtcgcgatccggaaatgggaggcaagccacagcgccggaaaaacccgcag |
| | gaaggcttgtacaacgaacttcagaaagataaaatggcagaagcatactccgaaatagggatgaa |
| | aggtgaacggcggcgaggcaagggccacgacggtctgtaccaagggttgtcaacggcaactaa |
| | agacacgtatgatgcacttcatatgcaagctctgccacccaggtga |
| SEQ ID NO: 24 | atgctgctgctggtgacaagcctgctgctgtgcgaactgccccatcccgccttcctgctgattcctc |
| | aggtccagcttgtgcaaagcggaggaggagtggtacagcctggccgctctttgagactgtcttgt |
| | gcggccagtggatttacattctcttcttatgggttgcattgggtcagacaagcaccgggcaaaggat |
| | tggaatgggtcgcggccattagctatgatggctcaaagaaatattatgccgattccgtaaaaggga |
| | ggttgacaataagccgggataacagcaagaacactttgtatcttcagatgaatagcctccgaccgg |
| | acgacacggcactgtatttttgcgcacgcgggtggtttgtagaacccctgagttggggacaaggta |
| | ctcttgtcacggtatcttctggcggaggtgggagtggtgggggtggcagtggcgggggtgggtc |
| | acaaagcgtgcttacacaacctccttctgcgagcggaactccgggacaacgggttacgatttcatg |
| | ctccggctcaagtagcaatataggatcaaatacagtgaattggtatcaacaactccctggcacagc |
| | gcccaagctgctgatctactctaataaccagaggccgagtggtgtgccagataggttcagtggctc |
| | taaatcaggtactagcgcgagcctcgccatttcaggacttcaatcagaggatgaagcggactacta |
| | ctgtgccgcgtgggatgattcacttaatggatatgttttcgggaccggaacaaaattgacggtattg |
| | gagagcaaatatggaccaccatgccctccatgtcctttttgggtcctggtggtcgtgggaggcgtg |
| | ctggcatgttattctctgctggtcacagtggctttcatcatcttctgggtcaagcgaggccggaagaa |
| | actgctgtacatcttcaaacagccttttatgcgcccagtgcagacaactcaggaggaagacggctg |
| | ctcttgtcggttccccgaggaagaggaagggggatgtgagctgcgcgtgaagttttctcgaagtg |
| | ccgatgctcctgcatatcagcagggacagaaccagctgtacaacgagctgaatctgggccggag |
| | agaggaatacgacgtgctggataagaggcgcggcagagacccagaaatgggcgggaagcca |
| | cgacggaaaaacccccaggaggggctgtataatgaactgcagaaggacaaaatggccgaggct |
| | tacagcgaaatcgggatgaagggagagagaaggcgcggaaaaggccacgatggactgtatca |
| | gggcctgagcactgccaccaaggacacctacgatgctctgcacatgcaggcactgccacccag |
| | gTGA |
| SEQ ID NO: 25 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCARGKRYFD |
| | YWGQGTMVTVSSGGGGSGGGGSGGGGSSYELTQPPSASGTP |
| | GQRVTISCSGGSSNIGSNTVNWFRQLPGTAPKLLVYFNNQRPS |
| | GVPDRFSGSKSGTSASLAIGGLQSDDEADYYCVAWDDSLNAP |
| | VFGGGTKVTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPR |
| SEQ ID NO: 26 | ATGCTGCTGCTGGTGACAAGCCTGCTGCTGTGCGAACTGCC |
| | CCATCCCGCCTTCCTGCTGATTCCTGAGGTGCAGCTGTTGG |
| | AGTCTGGGGGAGGCTTGGTACAGCCTGGGGGGTCCCTGAG |
| | ACTCTCCTGTGCAGCCTCTGGATTCACCTTTAGCAGCTATG |
| | CCATGAGCTGGGTCCGCCAGGCTCCAGGGAAGGGGCTGGA |
| | GTGGGTCTCAGCTATTAGTGGTAGTGGTGGTAGCACATACT |
| | ACGCAGACTCCGTGAAGGGCCGGTTCACCATCTCCAGAGA |
| | CAATTCCAAGAACACGCTGTATCTGCAAATGAACAGCCTG |
| | AGAGCCGAGGACACGGCCGTGTATTACTGTGCGAGAGGAA |
| | AGCGATACTTTGACTACTGGGGCCAGGGGACAATGGTCAC |
| | CGTCTCGAGTGGTGGGGGGGGCAGCGGTGGTGGAGGCTCT |
| | GGTGGAGGAGGGAGCTCCTATGAGCTGACTCAGCCACCCT |
| | CAGCGTCTGGGACCCCCGGGCAGAGGGTCACCATCTCTTGT |
| | TCTGGAGGCAGCTCCAACATCGGAAGTAATACTGTAAACT |
| | GGTTCCGGCAGCTCCCAGGAACGGCCCCCAAACTCCTCGTT |
| | TATTTTAATAATCAGCGACCCTCAGGGGTCCCTGACCGATT |
| | CTCTGGCTCCAAGTCTGGCACCTCGGCCTCCCTGGCCATCG |
| | GTGGGCTCCAGTCTGACGATGAGGCTGACTATTACTGTGTA |
| | GCATGGGATGACTCTCTGAATGCTCCGGTGTTCGGCGGAG |
| | GGACCAAGGTCACCGTCCTAGAGAGCAAATATGGACCACC |
| | ATGCCCTCCATGTCCTTTTTGGGTCCTGGTGGTCGTGGGAG |
| | GCGTGCTGGCATGTTATTCTCTGCTGGTCACAGTGGCTTTC |
| | ATCATCTTCTGGGTCAAGCGAGGCCGGAAGAAACTGCTGT |
| | ACATCTTCAAACAGCCTTTTATGCGCCCAGTGCAGACAACT |
| | CAGGAGGAAGACGGCTGCTCTTGTCGGTTCCCCGAGGAAG |
| | AGGAAGGGGGATGTGAGCTGCGCGTGAAGTTTTCTCGAAG |
| | TGCCGATGCTCCTGCATATCAGCAGGGACAGAACCAGCTG |
| | TACAACGAGCTGAATCTGGGCCGGAGAGAGGAATACGACG |
| | TGCTGGATAAGAGGCGCGGCAGAGACCCAGAAATGGGCG |
| | GGAAGCCACGACGGAAAAACCCCCAGGAGGGGCTGTATA |
| | ATGAACTGCAGAAGGACAAAATGGCCGAGGCTTACAGCGA |
| | AATCGGGATGAAGGGAGAGAGAAGGCGCGGAAAAGGCCA |
| | CGATGGACTGTATCAGGGCCTGAGCACTGCCACCAAGGAC |
| | ACCTACGATGCTCTGCACATGCAGGCACTGCCACCCAGG |
| SEQ ID NO: 27 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCARGKRYFDYWGQGTMVTVSS |
| SEQ ID NO: 28 | SYELTQPPSASGTPGQRVTISCSGGSSNIGSNTVNWFRQLPGT |
| | APKLLVYFNNQRPSGVPDRFSGSKSGTSASLAIGGLQSDDEAD |
| | YYCVAWDDSLNAPVFGGGTKVTVL |
| SEQ ID NO: 29 | EVQLLESGGGLVQPGGSLRLSCAASGFTFSSYAMSWVRQAPG |
| | KGLEWVSAISGSGGSTYYADSVKGRFTISRDNSKNTLYLQMN |
| | SLRAEDTAVYYCAKGKRYFDYWGQGTMVTVSS |
| SEQ ID NO: 30 | QSVLTQPPSASGTPGQRVTISCSGGSSDIGSNTVNWYQQLPGT |
| | APKLLIYYNNQRPSGVPDRFSGSKSGTSASLAISGLQSEDEAD |
| | YYCATWDDRMYSPVFGGGTKLTVL |
| SEQ ID NO: 31 | DVVMTQSPLSLPVTPGEPASISCRSSQSLVHSNRNTYLHWYLQ |
| | KPGQSPQLLIYKVSNRFSGVPDRFSGSGSGTDFTLKISRVEAED |
| | VGVYYCSQNTHVPPTFGQGTKLEIKGGGGSGGGGSGGGGSQ |
| | VQLVQSGAEVKKPGASVKVSCKASGYTFTDYEMHWVRQAP |
| | GQGLEWMGALDPKTGDTAYSQKFKGRVTLTADKSTSTAYM |
| | ELSSLTSEDTAVYYCTRFYSYTYWGQGTLVTVSS |
| SEQ ID NO: 32 | QVQLVQSGGGVVQPGRSLRLSCAASGFTFSSYGLHWVRQAP |
| | GKGLEWVAAISYDGSKKYYADSVKGRLTISRDNSKNTLYLQ |
| | MNSLRPDDTALYFCARGWFVEPLSWGQGTLVTVSSGGGGSG |
| | GGGSGGGGSQSVLTQPPSASGTPGQRVTISCSGSSSNIGSNTV |
| | NWYQQLPGTAPKLLIYSNNQRPSGVPDRFSGSKSGTSASLAIS |
| | GLQSEDEADYYCAAWDDSLNGYVFGTGTKLTVL |
| SEQ ID NO: 33 | gaggtgcagctgttggagtctgggggaggcttggtacagcctggggggtccctgagactctcctg |
| | tgcagcctctggattcacctttagcagctatgccatgagctgggtccgccaggctccagggaagg |
| | ggctggagtgggtctcagctattagtggtagtggtggtagcacatactacgcagactccgtgaag |
| | ggccggttcaccatctccagagacaattccaagaacacgctgtatctgcaaatgaacagcctgag |
| | agccgaggacacggccgtgtattactgtgcgagaggaaagcgatactttgactactggggccag |
| | gggacaatggtcaccgtctcgagtggtggggggggcagcggtggtggaggctctggtggagg |
| | agggagctcctatgagctgactcagccaccctcagcgtctgggacccccgggcagagggtcac |
| | catctcttgttctggaggcagctccaacatcggaagtaatactgtaaactggttccggcagctccca |
| | ggaacggcccccaaactcctcgtttattttaataatcagcgaccctcaggggtccctgaccgattct |
| | ctggctccaagtctggcacctcggcctccctggccatcggtgggctccagtctgacgatgaggct |
| | gactattactgtgtagcatgggatgactctctgaatgctccggtgttcggcggagggaccaaggtc |
| | accgtccta |
| SEQ ID NO: 34 | gaggtccagctgctggagagcggaggaggactggtgcagcctggaggaagtctgcgactgtca |
| | tgcgccgctagcggcttcaccttcagctcctatgcaatgagctgggtgcgacaggcaccaggcaa |
| | ggggctggagtgggtctccgctatctccggctctggaggctctacttactatgcagacagtgtgaa |
| | ggggcggttcacaatctccagagataactctaagaacactctgtacctgcagatgaactctctgag |
| | agctgaggacaccgcagtgtactattgcgccaagggcaaaaggtactttgattattggggacagg |
| | gcactatggtgaccgtctctagtggaggaggaggaagcggaggaggaggatccggcggagga |
| | ggcagtcagtcagtgctgacacagccacctagcgcctccggaaccccaggacagcgggtcaca |
| | atctcttgtagtgggggatcaagcgacattgggagcaacaccgtgaattggtatcagcagctgcct |
| | ggaacagctccaaagctgctgatctactataacaatcagaggccctccggcgtccctgatcgcttc |
| | tcaggcagcaaatccgggacttctgcaagtctggccattagtggcctgcagtcagaggacgaag |
| | ccgattactattgtgctacctgggacgataggatgtactctcccgtgttcggcgggggaacaaagc |
| | tgactgtcctg |
| SEQ ID NO: 35 | gatgtcgtgatgacgcagagccctctctctcttcccgttacccctggtgaacccgcatcaataagtt |
| | gccgctccagtcaatcacttgtacattcaaatcgcaatacctacctgcactggtatttgcagaagcc |
| | gggacaatcccctcaattgttgatatataaggtatccaatcgcttttctggagttcctgatagattcag |
| | cggatccgggtctggtactgatttcactctgaaaatatccagggtcgaagctgaggacgtaggcgt |
| | atattattgctctcagaacacgcatgtcccgccgactttcggccagggcactaaacttgagatcaag |
| | ggtggggggggcagcggtggtggaggctctggtggaggagggagccaggtccaactcgttca |
| | aagtggcgcagaggtcaaaaagccaggcgcgagcgttaaagtatcatgtaaggccagcggttat |
| | actttcactgattatgaaatgcactgggtgcgacaagcccccgggcaaggtcttgagtggatgggt |
| | gcacttgatccaaaaactggggatactgcctatagccagaaattcaaagggcgcgtcacactcact |
| | gccgacaaaagtacgagcacagcttatatggaattgagttcactgacgagcgaggatacggcagt |
| | ttattactgtacgcgcttctactcttacacttattgggggcaaggcactttggttactgtgtcctct |
| SEQ ID NO: 36 | caggtccagcttgtgcaaagcggaggaggagtggtacagcctggccgctctttgagactgtcttgt |
| | gcggccagtggatttacattctcttcttatgggttgcattgggtcagacaagcaccgggcaaaggat |
| | tggaatgggtcgcggccattagctatgatggctcaaagaaatattatgccgattccgtaaaaggga |
| | ggttgacaataagccgggataacagcaagaacactttgtatcttcagatgaatagcctccgaccgg |
| | acgacacggcactgtatttttgcgcacgcgggtggtttgtagaacccctgagttggggacaaggta |
| | ctcttgtcacggtatcttctggcggaggtgggagtggtgggggtggcagtggcgggggtgggtc |
| | acaaagcgtgcttacacaacctccttctgcgagcggaactccgggacaacgggttacgatttcatg |
| | ctccggctcaagtagcaatataggatcaaatacagtgaattggtatcaacaactccctggcacagc |
| | gcccaagctgctgatctactctaataaccagaggccgagtggtgtgccagataggttcagtggctc |
| | taaatcaggtactagcgcgagcctcgccatttcaggacttcaatcagaggatgaagcggactacta |
| | ctgtgccgcgtgggatgattcacttaatggatatgttttcgggaccggaacaaaattgacggtattg |
| SEQ ID NO: 37 | GFTFSSYAMS |
| SEQ ID NO: 38 | AISGSGGSTYYADSVKG |
| SEQ ID NO: 39 | GKRYFDY |
| SEQ ID NO: 40 | SGGSSNIGSNTVN |
| SEQ ID NO: 41 | FNNQRPS |
| SEQ ID NO: 42 | VAWDDSLNAPV |
| SEQ ID NO: 43 | SGGSSDIGSNTVN |
| SEQ ID NO: 44 | YNNQRPS |
| SEQ ID NO: 45 | ATWDDRMYSPV |
| SEQ ID NO: 46 | ATGCTGCTGCTGGTGACAAGCCTGCTGCTGTGCGAACTGCC |
| | CCATCCCGCCTTCCTGCTGATTCCTGAGGTGCAGCTGTTGG |
| | AGTCTGGGGGAGGCTTGGTACAGCCTGGGGGGTCCCTGAG |
| | ACTCTCCTGTGCAGCCTCTGGATTCACCTTTAGCAGCTATG |
| | CCATGAGCTGGGTCCGCCAGGCTCCAGGGAAGGGGCTGGA |
| | GTGGGTCTCAGCTATTAGTGGTAGTGGTGGTAGCACATACT |
| | ACGCAGACTCCGTGAAGGGCCGGTTCACCATCTCCAGAGA |
| | CAATTCCAAGAACACGCTGTATCTGCAAATGAACAGCCTG |
| | AGAGCCGAGGACACGGCCGTGTATTACTGTGCGAGAGGAA |
| | AGCGATACTTTGACTACTGGGGCCAGGGGACAATGGTCAC |
| | CGTCTCGAGTGGTGGGGGGGGCAGCGGTGGTGGAGGCTCT |
| | GGTGGAGGAGGGAGCTCCTATGAGCTGACTCAGCCACCCT |
| | CAGCGTCTGGGACCCCCGGGCAGAGGGTCACCATCTCTTGT |
| | TCTGGAGGCAGCTCCAACATCGGAAGTAATACTGTAAACT |
| | GGTTCCGGCAGCTCCCAGGAACGGCCCCCAAACTCCTCGTT |
| | TATTTTAATAATCAGCGACCCTCAGGGGTCCCTGACCGATT |
| | CTCTGGCTCCAAGTCTGGCACCTCGGCCTCCCTGGCCATCG |
| | GTGGGCTCCAGTCTGACGATGAGGCTGACTATTACTGTGTA |
| | GCATGGGATGACTCTCTGAATGCTCCGGTGTTCGGCGGAG |
| | GGACCAAGGTCACCGTCCTAGAGAGCAAATATGGACCACC |
| | ATGCCCTCCATGTCCTTTTTGGGTCCTGGTGGTCGTGGGAG |
| | GCGTGCTGGCATGTTATTCTCTGCTGGTCACAGTGGCTTTC |
| | ATCATCTTCTGGGTCAAGCGAGGCCGGAAGAAACTGCTGT |
| | ACATCTTCAAACAGCCTTTTATGCGCCCAGTGCAGACAACT |
| | CAGGAGGAAGACGGCTGCTCTTGTCGGTTCCCCGAGGAAG |
| | AGGAAGGGGGATGTGAGCTGCGCGTGAAGTTTTCTCGAAG |
| | TGCCGATGCTCCTGCATATCAGCAGGGACAGAACCAGCTG |
| | TACAACGAGCTGAATCTGGGCCGGAGAGAGGAATACGACG |
| | TGCTGGATAAGAGGCGCGGCAGAGACCCAGAAATGGGCG |
| | GGAAGCCACGACGGAAAAACCCCCAGGAGGGGCTGTATA |
| | ATGAACTGCAGAAGGACAAAATGGCCGAGGCTTACAGCGA |
| | AATCGGGATGAAGGGAGAGAGAAGGCGCGGAAAAGGCCA |
| | CGATGGACTGTATCAGGGCCTGAGCACTGCCACCAAGGAC |
| | ACCTACGATGCTCTGCACATGCAGGCACTGCCACCCAGGG |
| | GTAGCGGCGAGGGCAGAGGAAGTCTTCTAACATGCGGTGA |
| | CGTGGAGGAGAATCCCGGCCCTATGGGTCGGGGGCTGCTC |
| | AGGGGCCTGTGGCCGCTGCACATCGTCCTGTGGACGCGTAT |
| | CGCCAGCACGATCCCACCGCACGTTCAGAAGTCGGTTAAT |
| | AACGACATGATAGTCACTGACAACAACGGTGCAGTCAAGT |
| | TTCCACAACTGTGTAAATTTTGTGATGTGAGATTTTCCACC |
| | TGTGACAACCAGAAATCCTGCATGAGCAACTGCAGCATCA |
| | CCTCCATCTGTGAGAAGCCACAGGAAGTCTGTGTGGCTGTA |
| | TGGAGAAAGAATGACGAGAACATAACACTAGAGACAGTTT |
| | GCCATGACCCCAAGCTCCCCTACCATGACTTTATTCTGGAA |
| | GATGCTGCTTCTCCAAAGTGCATTATGAAGGAAAAAAAAA |
| | AGCCTGGTGAGACTTTCTTCATGTGTTCCTGTAGCTCTGAT |
| | GAGTGCAATGACAACATCATCTTCTCAGAAGAATATAACA |
| | CCAGCAATCCTGACTTGTTGCTAGTCATATTTCAAGTGACA |
| | GGCATCAGCCTCCTGCCACCACTGGGAGTTGCCATATCTGT |
| | CATCATCATCTTCTACTGCTACCGCGTTAACCGGCAG |
| SEQ ID NO: 47 | MLLLVTSLLLCELPHPAFLLIPEVQLLESGGGLVQPGGSLRLSC |
| | AASGFTFSSYAMSWVRQAPGKGLEWVSAISGSGGSTYYADS |
| | VKGRFTISRDNSKNTLYLQMNSLRAEDTAVYYCARGKRYFD |
| | YWGQGTMVTVSSGGGGSGGGGSGGGGSSYELTQPPSASGTP |
| | GQRVTISCSGGSSNIGSNTVNWFRQLPGTAPKLLVYFNNQRPS |
| | GVPDRFSGSKSGTSASLAIGGLQSDDEADYYCVAWDDSLNAP |
| | VFGGGTKVTVLESKYGPPCPPCPFWVLVVVGGVLACYSLLVT |
| | VAFIIFWVKRGRKKLLYIFKQPFMRPVQTTQEEDGCSCRFPEE |
| | EEGGCELRVKFSRSADAPAYQQGQNQLYNELNLGRREEYDV |
| | LDKRRGRDPEMGGKPRRKNPQEGLYNELQKDKMAEAYSEIG |
| | MKGERRRGKGHDGLYQGLSTATKDTYDALHMQALPPRGSG |
| | EGRGSLLTCGDVEENPGPMGRGLLRGLWPLHIVLWTRIASTIP |
| | PHVQKSVNNDMIVTDNNGAVKFPQLCKFCDVRFSTCDNQKS |
| | CMSNCSITSICEKPQEVCVAVWRKNDENITLETVCHDPKLPYH |
| | DFILEDAASPKCIMKEKKKPGETFFMCSCSSDECNDNIIFSEEY |
| | NTSNPDLLLVIFQVTGISLLPPLGVAISVIIIFYCYRVNRQ |

What is claimed is:

1. A method of treating cancer, comprising:
administering to a subject in need thereof a cell, wherein the cell comprises
a) a chimeric antigen receptor (CAR), wherein the CAR comprises an antigen-binding domain specific for Glypican-3 (GPC3), and
b) an armoring molecule,
wherein the antigen-binding domain comprises a scFv comprising a heavy chain variable region (VH) and a light chain variable region (VL),
wherein the VH comprises a CDR1 comprising the amino acid sequence of SEQ ID NO: 37, a CDR2 comprising the amino acid sequence of SEQ ID NO: 38, and a CDR3 comprising the amino acid sequence of SEQ ID NO: 39, and wherein the VL comprises a CDR1 comprising the amino acid sequence of SEQ ID NO: 40, a CDR2 comprising the amino acid sequence of SEQ ID NO: 41, and a CDR3 comprising the amino acid sequence of SEQ ID NO: 42,
wherein the armoring molecule is a dominant-negative TGF-β receptor type 2 (TGFβRIIDN) comprising amino acids 477 to 671 of SEQ ID NO: 47, and
wherein the cancer is GPC3 positive.

2. The method of claim 1, wherein the VH comprises the amino acid sequence of SEQ ID NO: 27.

3. The method of claim 1, wherein the VL comprises the amino acid sequence of SEQ ID NO: 28.

4. The method of claim 1, wherein the anti-GPC3 chimeric antigen receptor (CAR) and TGFβRIIDN armoring molecules comprise the amino acid sequence of SEQ ID NO: 47

5. The method of claim 1, wherein the armoring molecule counters immunosuppression of the cell in a tumor microenvironment of the cancer.

6. The method of claim 1, further comprising inhibiting tumor growth, inducing tumor regression, and/or prolonging survival of the subject.

7. The method of claim 1, wherein the cell is an autologous cell.

8. The method of claim 7, wherein the autologous cell is selected from the group consisting of a T cell, a Natural Killer (NK) cell, a cytotoxic T lymphocyte (CTL), and a regulatory T cell.

9. The method of claim 1, wherein the cancer is a solid tumor.

10. The method of claim 9, wherein the cancer is hepatocellular carcinoma, non-small cell lung cancer, ovarian cancer, and/or squamous cell lung carcinoma.

11. The method of claim 1, wherein the cancer is hepatocellular carcinoma.

12. The method of claim 1, further comprising administering to the subject an anticancer antibody and/or a chemotherapeutic.

13. The method of claim 1, wherein the scFv is encoded by the nucleic acid sequence of SEQ ID NO: 33.

14. The method of claim 1, wherein the CAR and armoring molecule are encoded by SEQ ID NO: 46.
